(19) United States
(12) Patent Application Publication
(10) Pub. No.: US 2004/0029135 A1
(43) Pub. Date: Feb. 12, 2004

(54) Diagnostic assays for BCWA

(76) Inventor: Elliot R. Ramberg, Hollywood, FL (US)

Correspondence Address: BUCHANAN INGERSOLL, P.C., ONE OXFORD CENTRE, 301 GRANT STREET, 20TH FLOOR, PITTSBURGH, PA, 15219, US

(21) Appl. No.: 10/319,068

(22) Filed: Dec. 13, 2002

Related U.S. Application Data
(60) Provisional application No. 60/340,669, filed on Dec. 14, 2001.

(51) Int. Cl. C12Q 1/68; G01N 33/567

(52) U.S. Cl. 435/6; 435/7.21

(57) ABSTRACT

A method of detecting a target analyte comprising the steps of providing a sample suspected of having a target analyte, protecting a specific target analyte, eliminating non-specific analytes, and detecting the presence of target with a signal.

### FIELD OF THE INVENTION

The present invention is directed to methods and compositions for detecting pathological conditions. In particular, the invention comprises methods and compositions using biological factors, such as complement components, for detecting pathological conditions.

### BACKGROUND OF THE INVENTION

Diagnostics has traversed a broad range of disciplines from an initial foothold in serologic diagnostics to DNA molecular diagnostics, such as those using PCR. Problems with many current diagnostic technologies include the inability to directly detect species specific mRNA and proteins, and many also lack specificity and sensitivity. The problems of detection of biological and chemical warfare agents, detection of molecular cancer metastasis, detection of residual disease, the early detection of HIV and other viral agents, sensitive carcinogen detection, sensitivity in detection of pathologic proteins or cells in normal tissue, and the need for heightened specificity and sensitivity in the determination of the precancerous state of dysplasia, illustrate the need for more accurate, sensitive and specific assays. Furthermore, most of these assays fail in detection of very low numbers of antigen or analyte targets, such as low number DNA, mRNA, protein or cellular targets in the presence of a large amount of non-specific material such as genomic DNA, mRNA, protein, or cells.

Another area of extreme importance in early infection or exposure diagnostics is the use of diagnostic processes in the detection and exposure to biological and chemical warfare agents, for the sake of simplicity in description these agents will hereafter be referred to as BCWA.

What is needed are methods and compositions that will detect the BCWA in a large sample of plasma or any environmental sample and concentrate and collect the pathologic BCWA targets in a small volume. Furthermore, methods-are needed for diagnostic assays that will detect the presence of the BCWA in vivo or ex vivo with high levels of sensitivity.

BCWA diagnostic technology should be configured to satisfy and provide the absolute requirements for high specificity (no false positive analysis), high sensitivity (no false negative analysis) and the identification of the very earliest stage in the infection or exposure time-course.

These factors are important in any clinical diagnostic test, but are exceptionally important in the detection of biological and chemical agent exposure due to the utter seriousness of its consequence on massive population segments.

It is understood that a diagnostic process to provide value, must perform at near perfect levels of specificity and sensitivity and identify the earliest infection/exposure to the pathologic target, an event that precedes the onset of clinical symptomology in order to afford the patient of available cure scenarios and lessen the impact of the disease on both the patient and the health care system that ultimately pays the involved costs. To achieve these three absolutely required goals, the present invention set out to raise their standards in diagnostic process design, and is presented as the concept of Haystack Processing.

All are aware of the analogy of the needle in the haystack, where the lesson learned is that a single needle cannot be detected in analysis of a single pinch of hay, but only by analysis of the entire haystack. Unfortunately, few relate this concept to diagnostic testing, wherein the current trend is in analysis of smaller and smaller pinches of hay to detect very low copy number targets, such as in PCR or related technologies. Their approach is unequivocally flawed.

Instead of reiteration of the well-documented flaws of small sample analyte analyses, and the inherent flaws in PCR analysis, a “Unified Approach” to diagnostic process design will now be presented.

Herein, one envisions four sample analyte haystacks appropriate for diagnostic testing:

I. Nucleic Acid—DNA

II. Nucleic Acid—RNA (mRNA, rRNA, tRNA, and viral RNA)

III. Whole Cells with unique surface expressed markers

IV. Soluble Peptides, proteins, and immunogenic chemicals

Haystack I: DNA Analysis

The present invention configures DNA analysis, not by testing very small DNA samples (1 microgram or less as in PCR), but by designing technology that will analyze thousands of times larger samples, the entire Haystack.

In Haystack Processing (HP), a single element is uniquely important in diagnostic assay design, namely the concept of non-specific target elimination (NTE). NTE compliance can be configured by use of enzymes that selectively destroy non-specific DNA (non-target DNA) analyte post protection of the target DNA by formation of a target DNA triplex structure (three DNA strands). The proven rationale is that Exonuclease III specific to double stranded DNA in combination with another Exonuclease specific for single strand DNA would in concert degrade all non-specific target DNA analyte, insuring increased specificity of the diagnostic process while the triplex protected target is refractory to the enzymatic degradation. In fact, all of the HP processes, such as, Target Protection Assay including Triplex Protection Assays (TPA) configurations (see related documents) possess multiple specificity levels designed into the assay process, thus satisfying the NTE requirement.

Furthermore, the enhanced sensitivity of the DNA assay, as well as the detection of the infection or exposure event at its earliest stage in the time-course is guaranteed by the processes' capability to analyze large amounts of DNA sample analyte (the entire haystack is tested, i.e. milligram or greater quantities of DNA) as well as the use of a sensitive chemiluminescent signal, in one embodiment, to detect the presence of very low copy number DNA targets in the sample. No signal amplification is necessary for detection of very low numbers of targets in a large sample.

These criteria define the concept of DNA analysis that is referred to as Haystack Processing.

Haystack II: Direct RNA Analysis

The present invention configures direct RNA analysis, not by testing very small RNA samples (1 microgram or less as in RT-PCR), but by designing technology that will analyze thousands of times larger samples, the entire haystack, accompanied by the use of a sensitive detection signal.

NTE compliance can be configured, herein, by use of enzymes that selectively destroy non-specific RNA (non-target RNA) analyte post protection of the target RNA by formation of a heterotriplex structure, composed of a single strand RNA target and a specialized DNA hairpin that upon complexation forms a stable DNA-RNA-DNA triplex structure. The rationale is that a single strand Exoribonuclease or other similar enzyme would degrade all non-specific RNA but will not destroy the protected target RNA analyte, which is refractory to the enzymatic degradation, insuring the increased specificity of the diagnostic process.

In fact, all of HP's mRNA TPA configurations possess multiple specificity levels designed into the assay process, thus satisfying the NTE requirement. Furthermore, the enhanced sensitivity of the direct RNA assay (no reverse transcriptase step necessary), as well as the detection of the infection or exposure event at its earliest stage in the time-course is guaranteed by the analysis of large amounts of RNA sample analyte (the entire haystack is tested, i.e. milligram or greater quantities of RNA) as well as the use of a sensitive chemiluminescent signal, in one embodiment, to detect the presence of very low copy number RNA targets in the sample. No signal amplification is necessary for detection of very low numbers of targets in a large sample. These criteria define the concept of direct RNA analysis that is referred to as Haystack Processing.

Haystack III: Cell Analysis (Prokaryotic/Bacterial and Eucaryotic/Mammalian)

A different strategy needed to be invented and implemented to detect the presence of a low copy number pathologic or other cell subset in a large normal cellular population. NTE, it was reasoned, could be achieved by assuring generation of an amplified signal only by cells comprising the target cell subset, while no signal is produced by normal cell analyte. The technology developed was called Complement Mediated Signal Amplification (CMSA), see related documents, and was configured as a complement fixation assay that would rely upon the presence of a specific target cell surface protein, upon complexation with a monoclonal antibody, to fix and activate immune complement, and be detected by assay of activated and amplified numbers of any product of the complement cascade (the signal). In one embodiment the signal is C3a peptide production by activation of the Alternate Complement Pathway. In another embodiment the signal is C4a peptide production by activation of the Classical Complement Pathway. Both will be herein presented.

Normal cell analyte with exposure to the monoclonal antibody will not fix and activate complement and, hence, generate no signal. Thus, a large amount of cellular analyte would generate no detectable signal, and the unique target cell (in one embodiment) would theoretically generate an amplified number of signals (see Table I). This process is characterized by a theoretical sensitivity down to a single target cell. Accordingly, the presence of millions of normal cells is transparent to the assay result. These criteria define the concept of cellular analysis that is referred to as Haystack Processing.

Haystack IV: Soluble Peptide, Protein and Immunogenic Chemicals

A similar strategy, but different reagent, needed to be developed to achieve NTE in soluble immunogenic target analysis. NTE, it was reasoned, could be achieved by assuring generation of an amplified signal by the soluble immunogenic target, while having no signal, produced, by normal soluble analyte. It was understood that the full signal amplification of the complement fixation and activation event had a requirement for a lipid matrix. It was reasoned that introduction of antibody sensitized red blood cell membranes could satisfy this requirement. The membrane (stroma) would be sensitized by the monoclonal antibody specific for the pathologic target.

Complexation of the soluble target with the sensitized RBC stroma would form the classic antigen/antibody complex and therefore fix and activate the complement cascade (complement fixation assay). The associated lipid stroma would assist in the full extent of complement signal generation by providing closely adjacent IgG molecules to support complement fixation, and by providing a lipid matrix to deposit amplified numbers of C4b which lead to C3 convertase (C1, 4b, 2a). This technique has been named, Membrane Assisted Complement Mediated Signal Amplification (MACMSA).

Normal soluble analytes with no specificity to the target antibody would not fix or activate complement and, hence, generate no signal. Thus a larger number of soluble analytes (millions) would generate no detectable signal and each unique soluble target analyte (in all embodiments) would generate amplified numbers of signals. This process is characterized by a theoretical sensitivity down to hundreds of molecules (see Table I). Accordingly, the presence of millions or more normal non-specific analytes is transparent to the assay result. These criteria define and fulfill the concept of soluble immunogenic target analysis that is referred to as Haystack Processing.

What is needed are methods and compositions that recognize the presence of very low numbers of infectious or other targets in an excess of non-specific, non-target or normal material. The target may be nucleic acid, such as DNA and RNA, cellular, or protein, in nature. Ideally, these methods and compositions comprise diagnostic technology that supports high levels of specificity and sensitivity in testing procedures. Preferred methods and compositions comprise diagnostic tests that are configured for early detection of the pathologic agent or other target in the sample by examining large amounts of sample analyte, including the pathologic targets, namely the DNA, RNA, cell, or soluble protein in solution, to detect the pathologic target earlier in the infection time-course or the BCWA targets in the exposure/infection time-course.

### SUMMARY OF THE INVENTION

The present invention is directed to methods and compositions for detecting pathological conditions. In particular, the invention comprises methods and compositions using biological factors, such as complement components, for detecting pathological conditions. Another invention comprises the use of DNA oligonucleotide hairpin probes to protect and capture the target for detecting pathological conditions. Particularly described are assays for non-specific target elimination that allow for detection of low copy number targets in a large field of non-target material. Such assays comprise methods comprising CMSA and MACMSA, which preferably comprise detection of complement proteins and components. Such assays also compromise methods of RNA-TPA, which preferably comprise detection of RNA target molecules. The assays of the present invention can be used for detection of changes in cellular molecules or nucleic acids that are part of disease states, infections, exposure to BCWAs or that can be used for detection of BCWA molecules in the environment.

The molecular (DNA or RNA) identification of pathologic targets has been presented in related documents and is called RNA-TPA. As presented in Table III, most pathologic microbes (bacterial and viral) can be detected in parallel by two different methods. One method, referred to as RNA-TPA, supports direct RNA analysis, another method, CMSA or MACMSA is also presented in related documents. Since both are independent methods that can be performed simultaneously, their results will not only indicate the BCWA exposure or presence in an environmental sample, but also confirm this event.

Not only do the methods and compositions of the present inventions comprise detection of nucleic acid and other molecular targets, but the methods and compositions of the present invention comprise diagnostics at supramolecular levels to confirm the presence of the pathologic or other cellular targets in tissues. One invention comprises the analysis of only the cell subset of interest in a very large cell specimen and has the ability to compartmentalize and assay each cell component for the analyte of interest. Other embodiments comprise target analyte sorting and separation from non-specific analyte for increased sensitivity of detection. CMSA comprises the fixation and activation of complement by interactions between cell subset specific surface membrane proteins, and monoclonal or other antibodies. The subsequent complement fixation process results in one embodiment in the production of the C3a peptide in quantities directly proportional to the extent of complement fixation, and in another, the C4a peptide.

One embodiment of CMSA, called MACMSA, comprises use of a soluble immunogen found in the cytoplasm or released into the cellular environment. These methods and compositions are used to diagnose the presence of pathologic or other specific soluble immunogens in the cytosol or those released into the surrounding media. The diagnostic assays of the present inventions are able to accurately diagnose the presence of the BCWA target and also determine the position of the patient in the time-course of the exposure infection, or other process.

### DESCRIPTION OF THE FIGURES

Table I represents the stoichiometry of C3a peptide production by the fixation of a single molecule of complement by the Classical Pathway. The Classical Pathway is described in “The Third Component of Complement Chemistry and Biology” (edited by John D. Lambris, Ph.D.) Basel Institute for Immunology, Grenzacherstr. 487, CH-4005 Basel, ISBN: 3-540-51513-5 and ISBN: 0-387-51513-5, Library of Congress Catalog Card Number 15-12910 (Springer-Verlag Berlin Heidelberg 1990).

Table II represents a side-by-side comparison of current state-of-the-art diagnostics (PCR) and MACMSA and mRNA TPA diagnostic processes in relation to their value in addressing the detection of the BCWA exposure event.

Table III represents a number of samples that can be taken to assess the occurrence of the BCWA exposure event.

Table IV represents a compilation of the CDC's categories and agents of biological warfare and indicates which of the currently discussed diagnostic inventions has value in detection of said agent.

Table V presents MACMSA and mRNA TPA diagnostic BCWA assays in relation to their theoretical sensitivities. In the MACMSA complement fixation assay two activated products of complement fixation C3a and C4a are compared.

Table VI sets forth the algorithm for AFB1 testing in tobacco processing.

Tables VII.1 to VII.5 represent the current EPA National Primary Drinking Water Standards involving the testing of regulated substances.

Table VIII.1 represents the government unregulated substances currently tested by the city of Albuquerque, N. Mex.

Tables IX.1 to IX.4 represent the Henry I. Stimson Center/Chemical and Biological Weapons Nonproliferation Project current description of biological weapons agents affecting man and anti-plant biological agents.

Table X represents the MACMSA signal used and the relative number of targets detected at multiple efficiencies using Alkaline Phosphatase (AP) labeling of the complement cascade product and assay by addition of 1,2 Dioxetane substrates.

Table XI represents a side-by-side comparison of MACMSA and PCR analysis of a range of bacterial contaminated water samples.

Table XII represents the current levels of regulated chemicals in drinking water supplies as parts per billion with comparison to levels datable by MACMSA analysis.

FIG. 1 represents the algorithm for the MACMSA BCWA assay.

FIG. 2a represents the algorithm for the RNA-TPA BCWA assay, namely the mRNA embodiment specific for use with microbes possessing mRNA.

FIG. 2b represents the algorithm for the RNA-TPA BCWA assay, namely the RNA embodiment specific for use with virus possessing RNA.

FIG. 3 represents the algorithm for the C3a MACMSA diagnostic assay to detect pathologic targets in 500 milliliters of plasma (platelet and leukocyte free), useful in protection of the blood supply.

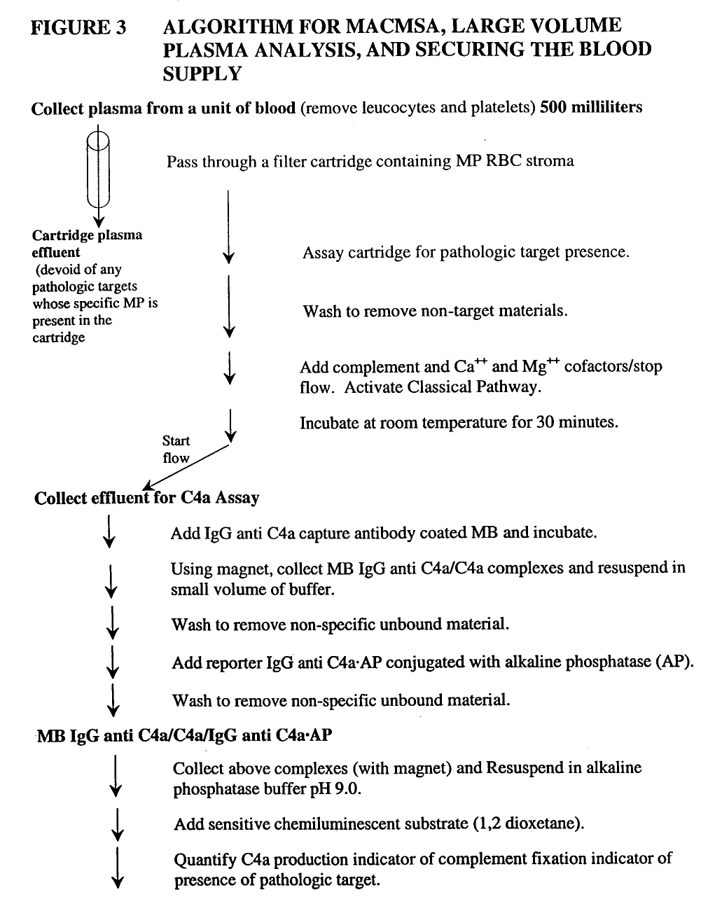

### DETAILED DESCRIPTION

The present invention comprises methods and compositions for the detection of low copy number targets of interest in diagnostic specimens in the presence of a large excess of normal material. The present invention can be used for diagnostic tests and has the capability to analyze specimens at the molecular (DNA and RNA), cellular, and tissue levels.

Methods and compositions of the present invention comprise non-specific target elimination, NTE (see related documents). NTE is used with processes that detect pathologic or other targets and supports high limits of specificity and sensitivity. Embodiments of NTE include the Haystack Processing technologies such as TPA (Target Protection Assay), RFTA (Restriction Fragment Target Assay), EAD (Enzyme Assisted Diagnostics) and CPA (Cutter Probe Assays), as described in U.S. Pat. Nos. 5,962,225, 6,100,040, and U.S. patent application Ser. No. 09/633,848, filed Aug. 7, 2000, PCT Application No. PCT/US98/24226, U.S. patent application Ser. Nos. 09/569,504, 09/443,633, and PCT Application No. PCT/US99/27525, each of which is incorporated herein in its entirety. The present invention is directed to methods and compositions including NTE, which comprise direct microbial RNA analysis by a method called RNA-TPA, see related documents, and Selective Target Monitoring technologies (STM) with Complement Mediated Signal Amplification (CMSA) and MACMSA (Membrane Associated Complement Mediated Signal Amplification). RNA-TPA is capable of sensitive and direct RNA analysis and is taught in U.S. patent application Ser. No. 09/443,633, U.S. Provisional Patent Application Nos. 60/226,823 and 60/325,442, and U.S. Provisional Patent Application filed on Sep. 21, 2001 (Applicant: Elliot R. Ramberg) entitled “Complement Mediated Assays for in vivo and in vitro Methods”, all of which are incorporated herein in their entirety. mRNA-TPA is taught in U.S. Patent application Ser. Nos. 09/776,568 and 09/933,307 and U.S. Provisional Patent Application Nos. 60/218,879 and 60/218,460, all of which are incorporated herein in their entirety.

Not only do the methods and compositions of the present inventions comprise detection of nucleic acid and other molecular targets, but the methods and compositions of the present invention comprise diagnostics at supramolecular levels to confirm the presence of the pathologic or other cellular target in tissues. STM functions on a cellular or nuclear level to negate the presence of normal cells or nuclei in the sample by the analysis of only the cell subset of interest in a very large cell specimen and has the ability to compartmentalize and assay each cell component for the analyte of interest. These low copy number analytes are detected at low copy numbers by generating a signal from the specific analyte of interest, while no signal occurs from the normal or non-specific analytes present in the compartment. Other embodiments of STM comprise target analyte sorting and separation from non-specific analyte for increased sensitivity of detection. STM on a cellular level comprises CMSA. CMSA comprises the fixation and activation of complement by interactions between cell subset specific surface membrane proteins, and monoclonal or other antibodies. The initiation of the complement fixation process in the presence of all complement proteins and cofactors, results in the production of all complement activation products in quantities directly proportional to the extent of complement fixation. Any other component or product of the fixed and activated complement cascade of proteins may be used as a signal, which will be presented later in this document and has already been presented in related documents.

CMSA is used for detection of target cells and supports NTE in any sample, particularly biological samples including, but not limited to, all body fluids, disaggregated cells, such as those derived from tissue samples, lymph nodes and fine needle aspirates, and environmental samples. An embodiment of CMSA analysis on a cellular level is taught in U.S. patent application Ser. No. 09/776,568, U.S. Provisional Nos. 60/218,460 and 60/226,825, and PCT/US 01/03649, all of which are incorporated herein in their entirety. For example, the intact cell, or cell membrane ghost, or nucleus is treated with a monoclonal antibody specific for a surface protein of interest, thereby forming an Ab/Ag complex that fixes complement. In the presence of all the complement components, complement is activated to produce activation products, whose quantity is directly proportional to the number of target cells present. The target analyte comprises any cell subset, an HIV infected T-cell, a dysplastic cell, and a neoplastic cell or may also be a cell membrane or cell nucleus, as well as an immunogenic carcinogen, pathologic prion protein, or BCWA molecule.

In CMSA and MACMSA, complement activation products are produced due to the interactive presence of a lipid membrane containing a unique surface protein (immunogen), a monoclonal or polyclonal antibody, and the complement cascade components. The presence and quantification of the C3a peptide or C4a peptide, for example, produced may be achieved by any number of methods known to those skilled in the art and discussed herein or in related documents. The key to CMSA is the presence of a lipid membrane that functions to amplify production of the amplified products by the complement cascade components. The present invention contemplates the use of lipid membranes found within the sample or lipid membranes that are added to the sample to be analyzed.

The methods and compositions comprising Membrane Associated Complement Mediated Signal Amplification (MACMSA) are used for sensitive soluble protein (immunogen) analysis. In one embodiment of this method, see related documents, RBC sensitized stroma comprising antibody to the unique BCWA immunogenic epitope is attached to a RBC lipid membrane, and interacts with the target analyte molecules present in the sample. Presence of the specific target analyte causes an Ag/Ab reaction to occur at the surface of the lipid RBC membrane, which in the presence of the complement components results in the full amplification of activated, amplified product production by the complement cascade and sensitive confirmation of the presence of the immunogenic target analyte. MACMSA is capable of molecular confirmation of a cellular diagnostic result as is taught in U.S. patent application Ser. No. 09/776,568, U.S. Provisional Nos. 60/218,460 and 60/226,825, all of which are incorporated herein in their entirety.

Soluble protein or peptide targets or other immunogenic molecules, whether pathologic or not, can be analyzed by STM on a soluble cytoplasmic molecular level that is monitored by use of MACMSA. MACMSA can also sensitively detect protein/peptide targets in any body fluid or other liquid sample including environmental. Another function of MACMSA is to detect and monitor non-protein chemicals in solution that are immunogenic thereby fixing and activating complement via the Classical Pathway, and to detect and monitor polysaccharides or other related molecules that fix and activate complement via the alternative pathway. MACMSA is used for detection of soluble target molecules in any biological or environmental fluid sample including, but not limited to, all body fluids, any soluble protein fluid suspension, environmental fluids, and chemical and material processing fluids containing the soluble, chemical or microbial immunogenic target analyte.

Unique pathologic proteins or other immunogens at low molecule number in a vast excess of normal proteins are identified, using STM with high specificity and sensitivity. The specificity comes from the use of multiple specificity steps, and the sensitivity is supported by the minimization of signal background by non-specific target elimination in the fluid samples, either extracellular or intracellular, and generation of signal from all target molecules either intracellular or of exogenous target in a large sample of analyte.

Similarly, RNA-TPA can be used to detect numerous BCWAs, see Table IV. A complete description of RNA-TPA can be found in related documents. Triplex Protection Assay (TPA) not only satisfy NTE mandates, but also support the highest levels of sensitivity assured by the ability to haystack process, namely analyze very large nucleic acid samples.

Selective Target Monitoring (STM):CMSA and MACMSA Analysis

STM cellular diagnostic technologies function on a cellular or nuclear membrane level to diagnose the presence of a pathologic or other cellular target, usually a cell or nuclear subset. A preferred embodiment comprises use of CMSA methods for signal amplification for the sensitive detection of the pathologic cell or nucleus. CMSA is based upon the activation and fixation of complement by addition to the target cell of an antibody specific to a cell surface or nuclear membrane protein. In eucaryotic cells, the classical complement activation pathway is activated and the extent and target presence monitored, in one embodiment, by production of the activated complement products. In prokaryotic cells, surface carbohydrates similarly participate by activation of the alternate complement fixation pathway also resulting in the production of the activated complement products. One embodiment of CMSA, called MACMSA, comprises use of a soluble immunogen found in the cytoplasm or released into the cellular environment. These methods and compositions are used to diagnose the presence of pathologic or other specific soluble immunogens in the cytosol or those released into the surrounding media. The diagnostic assays of the present invention are able to accurately diagnose the presence of the disease state and also determine the position of the patient in the time-course of the disease or other process (including BCWA exposure).

Signal amplification in STM on a cellular or nuclear level is directly proportional to the extent of complement fixation and activation. The cell surface membrane and nuclear membrane protein markers react with the specific monoclonal or other antibody to the immunogens resulting in fixation and activation of complement. Also cell surface polysaccharides and other materials fix and activate complement via the alternative pathway. The extent of complement fixation may be monitored in both complement pathways as a function of the number of activated complement products produced upon complement fixation, known to those skilled in the art.

RNA-TPA: Direct RNA Analysis to Detect BCWAs

RNA-TPA has been thoroughly presented in related documents and functions in accurate diagnostic detection of the pathologic RNA target. This is achieved by compliance with NTE edicts, which demand inactivation of non-target specific RNA from the sample to be tested.

This non-specific RNA is destroyed by Exoribonuclease or other Exonucleolytic enzyme functioning post protection of the target RNA species. This protection is achieved in one embodiment by the use of the DNA RP-TFO (reverse polarity-triplex forming oligonucleotide) hairpin, see related documents, which forms a stable triplex with the RNA target. The DNA hairpin possesses 8-aminopurine substituted bases that make the target/triplex stable at physiologic pH due to the additional Hoogsteen's bounding present. This renders the protected nucleic acid sequence (PNAS) of the target refractory to the Exonuclease treatment.

The PNAS is visualized by the use of a reporter probe that binds to the PNAS only. In one embodiment, a sensitive chemiluminescent substrate is used called a 1,2 dioxetane with a documented sensitivity of detection of 1000 molecules of AP or 1000 mRNA targets each hybridized with an AP labeled reporter probe. No target amplification or signal amplification is required in this direct RNA analysis process to achieve this level of sensitivity.

The exquisite sensitivity of the assay absolutely necessary for BCWA diagnostics is achieved solely by the ability to analyze a large amount of sample analyte (again related to Haystack Processing).

Membrane Assisted Complement Mediated Signal Amplification (MACMSA) and Target Signal Amplification

The methods and compositions comprising MACMSA comprise embodiments that function at the molecular level (DNA and RNA) by using compositions comprising attachment of an antigenic epitope or a peptide comprised of numerous epitopes to an oligonucleotide that acts as a reporter probe in nucleic acid assays. One embodiment of MACMSA, that can amplify a signal from a DNA reporter oligonucleotide (DNA and RNA target amplification, comprises using a single immunogenic epitope on a reporter probe to produce increased numbers of complement activation product molecules after binding to antibody sensitized RBC stroma sensitized by IgG anti epitope to the epitope in the presence of complement and the presence of additional IgG molecules in proximity on the stroma surface, followed by complement fixation and activation.

The extent of complement fixation and activation is influenced by many factors. These factors include avidity of the epitope and monoclonal antibody, and concentration of key intermediates in the complement cascade. For example, spiking native complement with additional components will increase the numbers of complement activation products produced by the presence of a single epitope in the assay. Other factors are determined by the method of complement fixation employed, either the Classical or Alternate Pathway and the relative effect of component spiking on complement fixation by each; and the use of sensitized RBC stroma used to amplify the activated component signal produced from a soluble immunogen, and methods of quantification of the resulting activation product. The factors influencing complement activation product production in MACMSA, when optimized, can provide significant amplified signal production per single target.

MACMSA and Single Target Immunogen Detection

To achieve the full signal amplification effect of a soluble protein or other immunogenic target in STM, a preferred embodiment requires the introduction of a lipid membrane to the assay namely antibody sensitized red blood cell stroma. The RBC stroma has a three-fold function in the assay, one, to collect the pathologic target, two, to concentrate the target, and three, provide a matrix to generate an amplified complement activation product signal, necessary to quantify low numbers of pathologic targets.

Production of Sensitized RBC Stroma

A preferred embodiment for production of RBC sensitized stroma employs the production of an IgG antibody pair, more preferably each IgG antibody has a different specificity. For example, one IgG of the pair is an IgG anti-D (Rh) monoclonal antibody used to attach the molecule pair (MP), and the antibody pair to the RBC surface, without any need for chemical modification of the RBC. The second IgG of the pair is an IgG anti-epitope monoclonal antibody used to bind the epitope present on the reporter probe or the BCWA or other immunogenic target and to promote fixation and activation of complement.

The red blood cells carrying the Rh determinants allow attachment of the antibody pair to the RBC membrane. A benefit of using the D (Rh) deterrminant is that the D/anti-D complex is known to those skilled in the art to not fix complement. Any other Ag/Ab pair that would not fix complement could also be employed in the methods and compositions of the present invention. RBCs with attached Ab pairs are referred to as sensitized.

The attachment of the MP to the D antigenic site on Rh POS red blood cells in a preferred embodiment calls for the use of Rh POS R₂R₂ RBCs. This Rh antigenic type offers the greatest expression on the RBC surface of any Rh type, will form a high avidity complex with MP and will, as previously stated, not fix complement.

The sensitized RBCs are washed and lysed in a hypotonic buffer solution or any other known method and the resulting membrane material is referred to as stroma. The stroma is washed to remove RBC contents and resuspended in a suitable buffer. The sensitized RBC stroma may now be used as a reagent.

In the MACMSA nucleic acid assay addition of stroma, the reporter probe with immunogenic epitope, fresh complement, and cofactors supports maximal activated product production. The solution may now be assayed for and activation product produced by use of any procedure known by those skilled in the art, such as sandwich and other ELISA and sensitized RBC lysis or any other method known to those skilled in the art.

In the MACMSA soluble immunogen (peptide, protein, or chemical) assay is similarly performed by addition of 1) sensitized stroma possessing an antibody possessing pathologic target specificity, 2) the pathologic target (BCWA) containing sample, and 3) fresh complement and cofactors, all of which will support maximal C3a production. The solution may be assayed for any complement activation product production by use of any procedure known by those skilled in the art, such as ELISA and sensitized RBC lysis (to be presented later) or any other method known to those skilled in the art.

Signal Amplification of Soluble Protein Targets in MACMSA

In these embodiments of STM, complement fixation and activation is quantified, for example, by a novel method, namely detection of production in the Classical Pathway of C4a activation product and in the Alternate Pathway of C3a activation product. Both are defined as ICPs and detection is achieved by assays for proteins or peptides that are known to those skilled in the art, including but not limited to, competitive and sandwich immunoassays such as ELISA assays, immunoMTRF, see related documents, or assays included in the present invention such as complement mediated signal amplification (CMSA) and lysis of sensitized RBCs, and lysis of liposomes containing fluorescence and quencher molecules.

Complement is a group of at least 25 glycoproteins with varying electrophoretic mobilities. Most circulate in the blood in an inactive precursor form and have effects in the body only after activation. Two major functions of complement in vivo are to promote the inflammatory response and to alter biological membranes to cause direct cell lysis or enhanced susceptibility to phagocytosis. Cell lysis occurs when antibody-mediated complement is fixed and activated by sequential interaction of the entire complement cascade. Most of these interactions result in the cleavage of an inactive protein with the release of small peptides in the complement response. In vitro, these peptides have no function, and may be called inactive complement peptides (ICPs). The peptides that do not participate in a direct complement response, meaning the lysis of cells or the opsonization of cells, are referred to herein as inactive complement peptides (ICPs). These inactive complement peptides (ICPs) have multiple in vivo functions: chemotaxis, enhancement of phagocytosis, alteration of vascular permeability, and stability of cell membranes (platelets and granulocytes). In a few instances, inactive proteins aggregate resulting in an active protein.

The Classical Complement Pathway Cascade:

The first complement component C1, attaches to the Fc portion of immunoglobulin molecules that have the appropriate binding site in the CH2 domain of the heavy chain. All mu (μ) chains have this site, and most gamma (y) chains. C1 is composed of 3 subunits: C1q, C1r, and C1s held together by calcium ions. If IgG is the type of antibody used, two adjacent protein antigenic sites, which exits on the antibody sensitized RBC stroma, must each bind an antibody molecule to form a doublet arrangement to provide the specific conformation for binding of the C1 complex. One IgM pentamer can bind the C1 complex. Clq binding to the Fe region of the antigen/antibody complex undergoes a conformational change that activates Clr, which in turn activates C1s, and forming C1 esterase that fixes complement and next cleaves C4 into antibody and membrane bound C4b and soluble C4a in solution. This reaction proceeds at high V max and the C1 esterase active enzyme is very stable, owing to the 10,000 molecules of C4a theoretically produced by each C1 esterase molecule present.

The following represent the steps in complement fixation and activation resulting in the production of the ICPs (C4a, C3a, and C5a).

Each molecule of C1q bound or fixed to the target membrane will produce at least an equivalent number of C3 convertase molecules and the ICPs, C4a, C3a, and C5a. At least one C3 convertase molecule is formed per one C1q molecule initially bound. Thousands of surface membrane proteins are expressed on a single cell, thus activation of complement fixed by multiple sites on a single cell or nuclear membrane can produce thousands of C4a, C3a, and C5a ICPs.

C1 esterase propagates the complement sequence by cleaving C4 into C4a and C4b and cleaving C2 to uncover a labile binding site. C4b contains a binding site and attaches to the cell membrane. C4a is released into the solution in vivo to stimulate anaphylaxis by stimulating mast cell degranulation and histamine release, thereby increasing vascular permeability. This released peptide may be used in a preferred embodiment of the present invention to amplify the signal from a target.

C2 attaches to the C4b molecule on the cell membrane. The larger fragment C2a combines with C4b to produce C4b2a, called C3 convertase, which possesses enzymatic activity. Each initial C1 esterase molecule can initiate attachment of hundreds of additional C4b which become C4b2a (C3 convertase) active complexes to the cell membrane in proximity to the C1q binding site (the lipid structure is a requirement for this event), and in doing so, releases additional C4a ICP which can be used for highly sensitive signal amplification methods in the present invention.

The third step, also an amplification reaction, is based on the function of all the bound C3 convertase molecules (C4b2a) to each cleave C3 molecules in solution resulting in release of additional C3a peptide fragments into the solution. This peptide has anaphylatoxin activity in vivo, and will be exploited as a signal amplification marker method in vitro. The upside to the use of C3a generation as a signal is the common production in both the Classical and Alternate Complement Cascade Pathways. The downside to the use of the C3a signal is the presence of a normal minimal C3a background due to a phenomenon called C3 tickover wherein C3 solution is normally cleaved at low levels by a hydration reaction of the C3 complement component in solution. The contribution of the C3 tickover to the C3a peptide level is much less than the C3a peptide levels generated by the presence of the target analyte in the Alternate Pathway and will result in the use of C3a production as a more sensitive signal for the complement fixation (and activation) assay in this pathway rather than in the Classical Pathway. The C3b larger fragment binds to the cell membrane complex or decays in solution. C3b fragments by themselves are not active catalytically and do not promote cell lysis but do increase phagocytosis upon attachment to the cell (opsonin activity in vivo). The importance here is the additional production and release of C3a into the solution in vitro and plasma in vivo.

Some C3b molecules join the extensive numbers of C3 convertase attached to the entire cell membrane forming C4b2a3b5b or C5 convertase releasing the C5a ICP into the solution. A further complication in the use of C5a as a signal lies in its additional production by the C3 convertase generated by the C3 tickover reaction. This limits the use of C5a as a signal to measure complement activation in the Classical Pathway.

In the presence of C5b, molecules of C6, C7, and C8 and a variable number of C9 molecules, assemble themselves into aggregates in the presence of Zn+2 called the membrane attack complex (MAC). The complex compromises the integrity of the cell membrane by altering permeability of the membrane and results in cell lysis.

The Alternate Pathway Complement Cascade

Cleavage of C3 and subsequent activation of the remainder of the complement cascade occurs independently of complement fixing antibodies. Cell surface particulate polysaccharide and lipopolysaccharide molecules, endotoxin, trypsin-like enzymes, and Ag/Ab complexes of IgA, and IgG4, that do not activate C1, all function to activate the Alternate Pathway. The activation is mediated by the cleavage of C3 into C3a, which is released in solution, and C3b. This molecule would be rapidly degraded in the fluid phase (Classical Pathway), but in the Alternate Pathway, C3b becomes stabilized by binding to the surface of a particulate activator of the Alternate Pathway called factor B, forming a stable C3b-factor B complex, itself interacting with a serum protease (factor D), cleaving factor B to produce C3bBb, that functions as a C3 convertase, again catalytically producing many additional C3a peptides (much greater than 100,000 C3a per bacterial target).

The Alternate Complement Activation Pathway is activated by few viruses, all bacteria, yeast or any other microbe containing polysaccharide or lipopolysaccharide elements in its exterior cell wall.

One embodiment of the present invention, the novel in vitro use of the complement cascade and the generation of the ICPs in the amplification of a signal to detect very low copy number of targets, is described herein.

Signal Amplification by Measure of Extent of Complement Fixation

The present invention comprises novel and sensitive methods for signal amplification, called CMSA and MACMSA. Activation of the complement cascade results in the production of tens of thousands of C4a peptides in the Classical Pathway and hundreds of thousands of C3a peptides in the Alternate Complement Pathway. Analysis of the sample for the detection and quantification of the ICPs results in the generation of >>100,000 C3a per pathologic prokaryotic microbe and eukaryotic cell or nuclear membrane, and generation of 10,000 C4a per soluble protein target or immunogenic epitope with the involvement of complement fixing Ag/Ab reactions in proximity to a lipid matrix (MACMSA).

Table I summaries the production of the ICPs and theoretical quantification provided by CMSA in the Classical Complement Pathway and the Alternate Complement Pathway.

Signal Amplification in the Classical Pathway

A preferred ICP is the peptide fragment C4a, because it is found in very high numbers after complement fixation and for additional reasons, previously herein stated and presented in related documents. Production of other ICPs (C3a, and C5a) may also be detected although they provide less signal amplification.

In general, the novel in vitro use of the complement cascade to quantify the presence of a pathologic cell or nucleus is based upon monitoring the extent of complement fixation and activation as a function of the number of inactive complement peptides (ICPs/C4a) that are produced. Basically, each target cell fixes thousand of complement molecules after addition of antibodies specific for the target cell surface protein and the subsequent reaction with the complement cascade. The initial complement molecules that are fixed can themselves exert an additional 10,000-fold amplification effect per antibody reaction with the target cell surface protein. This results in the following theoretical total signal amplification profile in CMSA:

a) Multiple cell surface protein markers (thousands) on the dysplastic cell each fixing complement, yielding 1000-fold signal amplification per pathologic target,

b) Primary 10,000-fold amplification during early stages of complement fixation, based on amplified C4a peptide production,

c) Total 10 million ICPs (C4a) produced per cellular or nuclear target.

In MACMSA, the following represents the total theoretical signal amplification profile:

a) A single soluble protein or reporter immunogenic epitope fixes one complement molecule.

b) Primary 10,000-fold amplification effect as a result of C4a production during early stages of single molecule complement fixation, similar to above, that is lipid membrane dependent requiring the use of the RBC sensitized stroma reagent.

c) Total 10,000 C4a molecules produced per target.

Signal Amplification in the Alternate Pathway

Methods of signal amplification using the Classical Complement Pathway employ methods of CMSCA and MACMSA. Signal amplification methods for the Alternate Pathway is similarly initiated by a step wherein a thioester on native C3 binds to polysaccharide, such as a polysaccharide on the surface of an organism. Next, the complex is stabilized by the binding of Factor B and its subsequent activation:

C3H₂O+Factor B+Factor D=C3bBb+C3a

C3bBb=activated Factor B or C3 convertase

The first signal amplification step occurs by the convertase cleaving numerous native C3 molecules producing numerous C3a peptides and additional C3b molecules that attach to the complex to form additional C3 convertase, that release additional C3a into the solution.

The C3 convertase (C3bBb) cleaves hundreds of C3 molecules generating additional C3b molecules, which attach to the complex and amplifies its activity. Cleavage of the C3 mediates release of hundreds of C3a ICP molecules to mediate amplification in vivo of the immune response and in vitro signal amplification.

The second level of signal amplification employs the aggregation on the surface of a microorganism or a protein aggregate of numerous C3b units, Factor B, and Properdin (stabilizing protein) acts as a potent C5 convertase producing hundreds of C5a (ICPs), thus cleaving C5 to an active C5b and release of a C5a into the solution. The remainder of the complement cascade is identical to later steps in the Classical Pathway. Thus, the ICPs, generated by complement fixation of the Classical Complement Pathway, or the Alternate Complement Pathway are used for in vitro signal amplification target detection strategies.

Detection and Quantification Assays for the ICPS (C4a, C3a, C5a)

Many assay strategies are available to determine the presence and quantification of the individual or combined ICPs. The present invention comprises assays for measuring the presence and number of individual or combined ICPs and is not limited to the assays and embodiments disclosed herein. The individual ICPs can be quantified by assays for proteins, including but not limited to sandwich ELISA assays, or similar assays that use a capture antibody bound to a solid support and a different labeled reporter antibody both specific for different epitopes on each ICP (C4a, C3a, C5a).

For example, an embodiment of the C3a sandwich ELISA assay is configured using a biotinylated anti-C3a reporter antibody and is followed by addition of an IgG anti-biotin alkaline phosphatase polymer conjugate to facilitate signal generation per C3a molecule by introduction of the substrate, 1,2-ioxetanes. Any other enzyme known to those skilled in the art may be used to quantify the number of C3a molecules. The enzyme may provide a color signal, a fluorescent signal, or a chemiluminescent signal, all known to those skilled in the art.

A preferred embodiment of the signal generated by the C3a peptide molecules is mediated by the use of an anti-biotin alkaline phosphatase polymer, known to generate 4 logs of signal per polymer molecule. The polymer is then reacted with a chemiluminescent substrate generating a stable light signal. One such substrate is the 1,2-Dioxetanes, which have been shown to detect 0.01 attomole quantities of alkaline phosphatase enzyme (1,000 molecules of enzyme), translating to a ten-fold increased level of target detection by the enzyme polymer. This detection system will support unprecedented high levels of target detection and, due to the nature of antibody conjugates to enzymes, will provide a relatively low background in the negative controls.

Such methods may also be automated. An example is shown below.

Step I. Prepare a magnetic bead with a covalently bound IgG anti-C3a capture antibody. The binding can be achieved by any chemistry known to those skilled in the art such as covalently linking an aminated magnetic bead to the carboxyl group on the c-terminal end of the antibody molecule, or any other chemistry known to those skilled in the art.

Step II. The magnetic bead is washed to remove non-bound capture probes and Step m. Conjugated beads are added to a sample containing the C3a peptide in solution, which is mixed and incubated.

Step IV. The magnetic beads are washed to remove non-specific bound materials

Step V. Addition of another antibody, IgG anti-C3a, which has reporter function and is specific for a different epitope on the C3a peptide molecule, similar to C4a. This antibody possesses an alkaline phosphatase (AP) polymer covalently attached to it. This may be generated by any method known to those skilled in the art, the preferred one being attachment to an antibody amine of the maleimide derivative of the AP polymer, which results in covalent bond formation. Any other chemistry may also be employed. Another embodiment might use a IgG anti C3a reporter antibody conjugated with the c-myc peptide, followed by use of the IgG anti c-myc/alkaline phosphatase conjugate. In this situation the AP is not in polymeric form.

Step VI. Wash to remove unbound reporter probe. The number of Washes and the wash buffer may be critical in resolving non-specific signal from unbound reporter enzyme.

Step VII. Addition of the magnetic beads to a solution containing the 1,2-Dioxetane substrate and incubate under conditions for the production of a stable chemiluminescent signal.

The reporter antibody, and hence the target, is detected by the activation of a chemiluminescent substrate to produce light by enzymatic catalysis.

The reporter antibody can also be detected using immunoMTRF methods as disclosed in U.S. patent application Ser. No. 09/443,633 or by conjugating a label, such as a single molecule of fluoroscein isothiocyanate, to each ICP reporter antibody.

Another method for assay of C3a production would be the use of IgG anti-C3a antibody imbedded on the surface of a liposome containing fluorescence and quencher molecules in close proximity, so that no fluorescent signal can be detected. Introduction of a C3a peptide to the antibody-sensitized liposome, in the presence of the complement components will result in complement mediated lysis of the liposome, releasing the fluorescence and quencher molecules into the solution. Their release and separation can be monitored by the detection of a fluorescent signal. The extent of liposome lysis is directly proportional to the quantity of ICPs produced and targets present.

Another method of the present invention for C3a quantification comprises steps to identify and quantify the specific ICP of interest using sensitized RBCs conjugated with anti-specific ICP antibodies that will only react with the free-floating ICPs in solution. In this embodiment RBCs linked to anti-ICP monoclonal antibodies in vitro will in the presence of complement undergo complement-mediated immunoerythrocyte lysis, releasing hemoglobin for quantitation. The extent of RBC lysis is directly proportional to the quantity of ICPs produced and targets present. This will be presented in detail later in this document.

Generation of Sensitized RBCs FOR C3a Assay: RBC Enzyme Treatments

One embodiment of the present invention comprises methods to identify and quantify specific ICPs of interest comprising use of sensitized RBCs that are conjugated with specific anti-ICP antibodies that will only react with the free-floating ICPs in solution and in the presence of fresh complement, result in red blood cell lysis upon binding of free ICPs with subsequent complement fixation and red blood cell lysis.

The sensitized or immunoRBCs can be generated by stripping the RBCs with a proteolytic enzyme such as bromelain, ficin, or papain and by other methods known to those skilled in the art, that attach the ICP specific antibodies to the RBC surface, producing sensitized immunoerythrocytes which bind the free floating ICP in solution. This attachment of an antibody to the stripped RBC surface by simple exposure of the antibody to the erythrocyte provides a non-covalent attachment of the antibody molecule, and is sufficient for some applications. Due to the fact that chemical modification of the RBC surface involves increased fragility of the modified RBC, which may result in the spontaneous release of hemoglobin and make quantification of the ICP peptides difficult, other methods are also contemplated by the present invention.

A novel process for production of antibody sensitized RBCs is mediated by the use of an IgG antibody pair. The characterization of the molecule is as follows:

1. Two IgG molecules are attached to each other by any method known to those skilled in the art, where the attachment does not interfere with the antibody binding sites.

2. One antibody must be specific to any of the ICP peptides for assay; for example, the IgG anti-C3a antibody used in the C3a peptide assay. Other embodiments require this antibody to be specific for any immunogenic epitope on the target.

3. The other antibody is specific for an antigen on the RBC. A most-preferred embodiment comprises use of an antibody specific for the Rh determinant. The Rh determinant extensively covers the RBC membrane with thousands of molecules and this is the site at which the antibody pair binds to the erythrocyte. This antigen/antibody reaction does not fix complement. This is important in light of the use of this immunoerythrocyte in the presence of fresh complement to monitor attachment of the C3a peptide to the complement fixing anti-C3a antibody in close proximity to the RBC surface. Any interactive antigen/antibody reaction that does not fix complement may also be employed and may involve the use of Fab fragments devoid of an intact Fc region as the attachment antibody.

4. The Rh determinants on the RBC surface are responsible for binding the antibody to the C3a and providing additional adjacent antibodies in close proximity to the lipid membrane surface without altering the stability of the immunoerythrocyte.

The sensitized immunoerythrocyte in the presence of the corresponding peptide and fresh complement will undergo lysis in vitro by the membrane attack complex and hemoglobin will be released, which may be quantified (presented later).

The Antibody Pair Method for in vivo Neutralization of a Pathologic Analyte by Sensitized RBCs

Another embodiment for use of the antibody-pair molecule may involve its use in vivo to neutralize the activity of a pathologic analyte such as BCWAs. This analyte may be a bacterium, bacterial toxin, yeast or fungus (or toxic product from), viral particle, antibody molecule, dysplastic or cancer cell, and even an immunogenic environmental carcinogen. Attachment of the pathologic target specific IgG anti-D antibody and the attachment antibody, namely, the molecule pair to the RBC surface would facilitate the immediate attachment and sometimes, simultaneous neutralization of the pathologic analyte by the attachment to any of the RBCs that have been sensitized.

Neutralization of the activity of the pathologic analyte would immediately block its reactive effect and would initiate its removal from the body mediated by macrophage phagocytosis or the function of another clearance system in the spleen and liver and other body sites. It is known to those skilled in the art that RBCs possessing immune complexes on their surface are rapidly cleared by these body systems.

Production of Sensitized RBC Stroma for use in MACMSA

MACMSA requires the interaction of a lipid/antibody (MP) complex with a soluble protein or reporter probe immunogenic epitope. The preferred embodiment for production of this complex is the sensitization of the RBCs by the aforementioned method with subsequent lysis of the sensitized RBCs in a hypotonic buffer solution resulting in the production of antibody attached lipid membrane (RBC stroma) that will exert the full signal amplification effect of the immunogenic epitope or soluble protein by the MACMSA process. Stroma production is achieved by placement of the immunoerythrocytes in a hypotonic buffer resulting in RBC lysis and membrane ghost formation. The stroma is then washed in buffer and resuspended in buffer for use as a reagent.

Defining the Characteristics of a Biological/Chemical Warfare Agent Exposure Diagnostic

The BCWA exposure event presents itself as a critical sequence of events whose outcome could range from minimally significant to totally catastrophic possible resulting in extensive mortality.

Several factors control the ultimate result from the BCWA exposure and place this result in the continuum from insignificant to catastrophic. The following is a list that directly dictates the result of the exposure event.

1. Selection of a common characteristic possessed by all agents of biological and chemical warfare,

2. Design of a diagnostic platform and process that will result in essentially 100% specificity,

3. Design of a diagnostic platform and process that will result in essentially 100% sensitivity,

4. Design of a diagnostic platform that will pinpoint the exact time of the BCWA exposure event.

Each of the aforementioned will be individually discussed in relation to the Unified Diagnostic Approach previously presented, with reference to some competitive diagnostic processes.

Selection of a Unique Characteristic of all BCWAs

The importance of development of a standard analysis platform and process, with the capability to program into the system, the specificity of the agent or agents to be detected, is dependent on selection of a common characteristic of all BCWAs.

The HP BCWA diagnostic processes has defined this characteristic as being the immunogenicity of all the BCWAs. Each BCWA has its own unique specificity that can be realized on the molecular (RNA) or supramolecular (antibody specific cell, biological toxin or chemical poison level). To select the appropriate BCWA diagnostic one must possess a unique DNA/RNA sequence for the biological agent or the biological toxin. One may also possess a monoclonal or other antibody or antibody fragment with specificity to the microbe, microbial toxin, or epitopes on the chemical poisons.

Table IV represents the CDC categorization of agents of biological warfare (the chemical agent list is too extensive to be presented but similar rules and diagnostic processes are shared in common by biological and chemical agent analytes).

Selection of a Diagnostic Process to Assure the Highest Levels of Specificity

As stated earlier in this document, the ability of any diagnostic to conform to the edicts of NTE will support the highest levels of specificity (referred to as having no false positive results). Furthermore, possession of a unique genetic sequence or monoclonal antibody to each BCWA further supports these high levels of specificity of the diagnostic process.

Table II characterizes and compares two HP technologies and the current industry standard, PCR. PCR due to primer specificity and amplicon confirmation via gel analysis provides adequate specificity, while HP's MACMSA and RNA-TPA provide equivalent or better specificity due to compliance with NTE edicts.

Selection of a Diagnostic Process to Assure the Highest Levels of Sensitivity

The sole factor that contributes to the sensitivity of the BCWA diagnostic assay is the ability to test very large amounts of sample analytes. Table III represents a list of different samples critical to the detection of BCWA exposure and represents the ability to analyze the appropriately sized sample to assure sensitive diagnostic results.

Table II characterizes HP technologies and the current industry standard, PCR. PCR suffers greatly by its inability to analyze sufficiently large samples to insure high sensitivity. Most diagnostic processes fall far short of possessing the capability to process very large to large sample sizes. All of HP invented processes including MACMSA and RNA-TPA were expressly designed to achieve such a capability and these diagnostic processes can truly be said to support the necessary high sensitivity absolutely required in any BCWA diagnostic process.

Selection of a Diagnostic Process to Detect and Pin-Point the Exposure Event

It is not difficult to understand the absolute necessity to detect BCWA exposure in a population group as soon after exposure as possible. This period very early in the BCWA exposure time-course provides key information necessary to manage the BCWA exposure event, and to initiate some treatment modality (vaccine, immunization, antibiotic or other drug) in an attempt to reduce population mortality.

Prompt detection of the BCWA exposure event and prompt therapeutic treatment will greatly reduce mortality, monetary, social, psychological, and other impacts of this deleterious event.

Most current diagnostic technologies cannot detect this early stage of the BCWA exposure event primarily due to their inability to process a clinically relevant sample size. All of HP processes test the entire haystack, while most others, like PCR, focus on analysis of a pinch of hay to find the elusive single needle (BCWA) in the haystack, a difficult if not impossible situation.

The key to MACMSA and RNA-TPA diagnostic success in detecting early stage BCWA exposure lies in their ability to process the entire haystack (discussed previously).

The Crucial Role of Sample Size in Prediction of Diagnostic Value of an Assay to the BCWA Exposure Event

In a routine diagnostic assay the direct goal would be to detect the infectious disease agent at any time prior to clinical symptomology. In normal infections time-courses, the pathologic agent is usually introduced by a low target exposure to the host followed by a reasonable time (week to month) to reach a critical pathologic target load in the body before the onset of clinical symptoms and threat to the well being of the affected host.

Unique to the BCWA exposure event is the potential for very large copy number BCWA exposure, which often may reach a critical pathogen load that is life-threatening in 24 hours or less, depending on the extent of BCWA exposure.

In this application the BCWA diagnostic must rapidly detect the exposure event by testing environmental and host samples with processes configured to provide the absolute highest levels of sensitivity possible. Herein, the exposure event is of tantamount importance to pinpoint the exact time of and to detect, due to the incredibly rapid onset of host BCWA pathologic target loads that almost immediately place the host in a life-threatening situation. This rapid attainment of a critical BCWA load to reach life-threatening status is promulgated by several factors:

Size of the BCWA exposure load,

The presence of virulence factors, microbial toxins, bacterial capsules, bacterial spores, and others that rapidly place the life of the affected host in jeopardy,

The confusion of minimal symptomology seen after the exposure event; The host experiences mild cold or flu symptoms (in bronchial exposure to the BCWAs) or minimal skin lesions (in cutaneous or subcutaneous exposure to BCWAs) seeming to pose little life threatening capability of the agent. This situation rapidly changes with the dramatic onset of severe life threatening systemic symptomology at a time where cure scenarios are bordering on worthlessness due to the exposure and infection kinetics of the BCWA agent.

The only answer to this dilemma is the earliest detection of the exposure event by any diagnostic process. This can be achieved as stated herein by analysis of large volumes of sample analyte. The following will present Haystack Processing diagnostic technologies MACMSA and RNA-TPA with emphasis on very large sample analysis.

Haystack Processing of very Large Samples to Detect the BCWA Exposure Event

Table III correlates sample volume analysis to the most accurate detection of the BCWA exposure event (represented by the highest sensitivity of the included assays, MACMSA and RP-TFO).

Herein, analysis of environmental samples play a key role in the BCWA exposure event due to their ability to detect BCWA exposure event even before their presence can be readily detected in the exposed host. Some environmental samples are presented and the largest sample analyte must be capable of being assayed in the BCWA diagnostic to detect very low levels of these BCWAs. These are water, soil, air or ingestibles. Furthermore, patient body fluids can also provide the samples for analysis and their large size also assures the highest levels of sensitivity of the BCWA diagnostic. These are blood, plasma, urine, cerebrospinal fluid (CSF), sputum, and nasal lavage fluid, as well as biopsy or skin scraping samples. Each large sample will represent the hay in the haystack, which will be completely analyzed by HP's BCWA diagnostic assays MACMSA and RNA-TPA.

The capability to analyze very large samples for the pathologic targets in MACMSA and RNA-TPA will now be discussed.

MACMSA Analysis of Environmental Haystacks: Importance of Minimal Manual Preprosessing Steps

Analysis of a large sample for the presence of the BCWA in MACMSA is initiated by a manual/semi-automated approach to collect and concentrate all BCWAs in the large environmental or host sample. This can be achieved by the use of magnetic beads and sensitized MP RBC stroma as previously discussed.

The molecule pair employed is the IgG anti BCWA-IgG anti-D (antibody pair), and the RBC is characterized as Rh POS R2R2, also previously discussed. To analyze very large sample volumes, the MP RBC stroma produced, see related documents, is attached to a magnetic bead by any method known to those skilled in the art.

In one embodiment the magnetic bead is coated with IgG anti CR1, which will bind the MP RBC stroma. In another embodiment, an AB fragment devoid of an Fe region is used to anchor the MP RBC to the magnetic bead. Any site other than the MP attachment site (D)) on the RBC surface may be used.

In essence, since the MACMSA diagnostic assay is dependent on complement fixation to detect BCWA presence, both attachment of the MP to the RBC and attachment of the MP RBC stroma to the magnetic bead must not fix complement. Complement in MACMSA must only be fixed by the interaction of the BCWA with the MP RBC stroma.

The liquid samples to be assayed, water, urine, CSF, and plasma can be directly analyzed by addition of a predetermined amount of magnetic beads (MB) with the attached MP RBC, wherein the MP is BCWA specific and the amount of MB/MP RBC added is directly proportionate to the sample size. The mixture then is incubated with agitation, in one embodiment by use of roller bottles to gently mix the additives without causing disruption of the MB/MP RBC/BCWA formed complex for a predetermined incubation time that is empirically determined as optimal (probably in the range of 30 to 60 minutes).

Another assay embodiment might involve use of a MP RBC filter cartridge that contains the BCWA specific antibody sensitized stroma, wherein the liquid sample is slowly run through the cartridge. Any other similar method may be used. This will be discussed in the blood plasma analysis example presented at the end of this document.

The solid samples (soil, ingestible foodstuffs, biopsy, skin lesion scrape material, or nasal lavage) must be diluted and mixed to solubilize the BCWA in the liquid layer, which when separated, is similarly processed as previously described.

In both liquid and solid sample analysis the MB/MP RBC/BCWA complex is easily collected by magnetic attraction of the complex, which is then resuspended in a small volume for subsequent automated assay.

Algorithm for MACMSA Analysis of Large Volume Samples

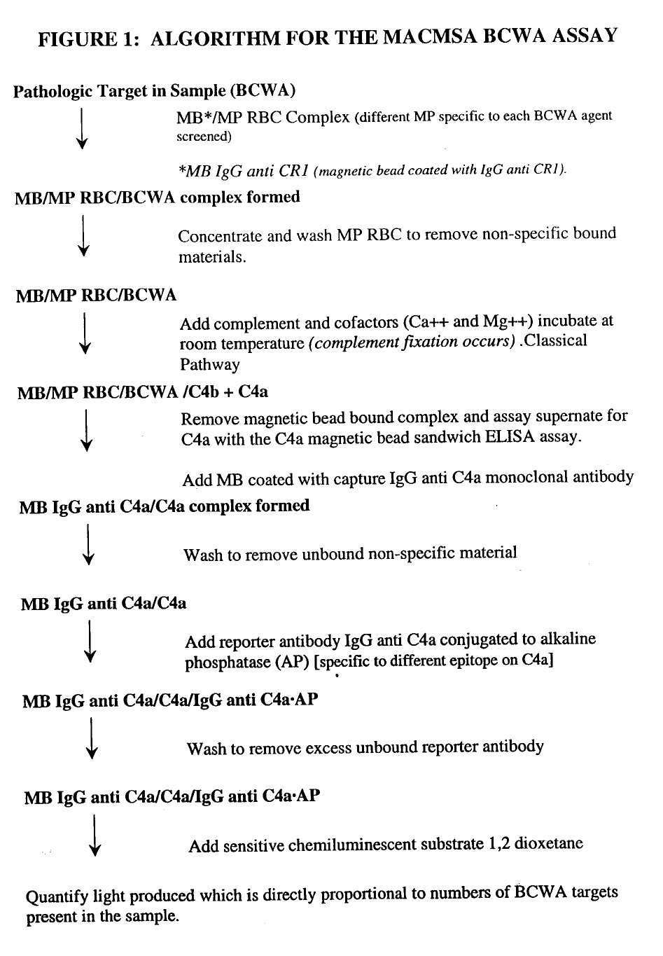

The following steps comprise the MACMSA BCWA complement fixation assay. See FIG. 1.

Step I: Concentration of the BCWA targets in a small sample volume. This step may be automated, semi-automated, or manual. The concentration step has been previously described.

Step II: The MB/MP RBC/BCWA complex is transferred manually or by automation to the AGENDA I robotic device of CyGene for continuation of the automated phase of the complement fixation assay.

Step III: The MB/MP RBC/BCWA complex is washed with buffer to remove non-specific materials (such as carbohydrates), which are known to fix complement.

Step IV: Complement reagent and cofactors are added to the MB/MP RBC/BCWA complexes and incubated at 37° C. (room temperature) for 15-30 minutes may be empirically determined as satisfactory). During which time, BCWA targets present fix and activate complement, resulting in C4a peptide production in the Classical Pathway and C3a production in the Alternate Pathway.

Step V: The MB/MP RBC/BCWA complexes are removed by the AGENDA magnets and the supernate containing the C3a and C4a peptides is assayed by any method known to those skilled in the art. In one embodiment a magnetic bead C3a and C4a peptide sandwich ELISA may be used.

Step VI: Perform the C4a peptide MB sandwich ELISA (same with modifications for C3a). Herein, MBs coated with a capture IgG anti C4a monoclonal antibody are added to the above supernate and incubated at room temperature for an empirically determined time.

Step VII: Remove the C4a attached magnetic beads and wash beads to remove non-specific material.

Step VIII: Transfer the C4a MB to another well containing the reporter antibody, IgG anti C4a, conjugated with alkaline phosphatase enzyme. Both the C4a capture and reporter monoclonal antibodies possess specificity to different epitopes on the C4a molecules.

Step IX: Wash the MP C4a complex to remove unbound reporter antibody.

Step X: Add a sensitive chemiluminescent substrate in one embodiment a 1,2 dioxetane substrate, with a documented sensitivity level of detection of 1000 AP molecules. Detection of 100-1000 BCWA targets (100% and 10% assay efficiency) is supported by this assay. Any other signal or signal amplification process, known to those skilled in the art, may be included in this assay.

Use of HP RNA TPA Diagnostic Process for the Early Detection of the BCWA Exposure Event

The Target Protection Assay, TPA (Triplex Protection Assay, see related documents) provides a very sensitive diagnostic assay to detect bacteria on the mRNA molecular level as well as the RNA of viral origin as the BCWA. Chemical agents could be detected on this nucleic acid molecular level only if the chemical directly reacts with nucleic acids, such as found with mutagenic chemical carcinogens, teratogens, and the like.

Currently, RNA TPA will be presented for the microbial agent possessing DNA, with an mRNA assay, and for the viral agent possessing RNA, with a viral RNA assay.

mRNA TPA for Microbial BCWA Detection

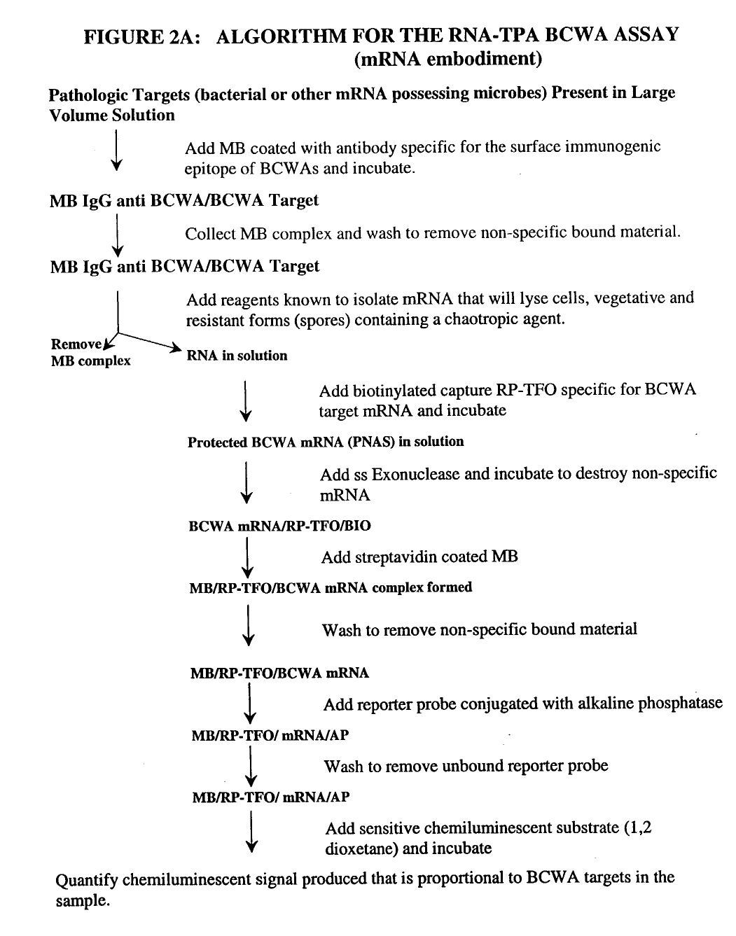

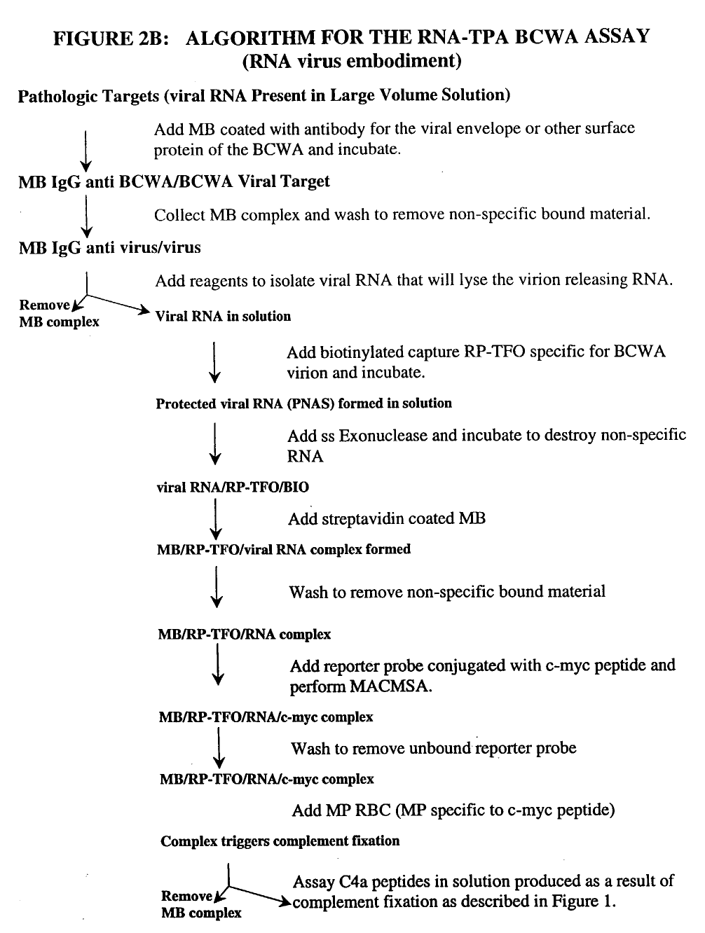

The process of mRNA TPA will be presented in FIG. 2. The steps are:

Step I: Concentration of microbial BCWA in a very large volume sample

Again, any sensitive diagnostic assay must process and analyze very large sample volumes as described. Magnetic beads coated with an antibody specific for the microbial pathogen to either a surface protein or surface carbohydrate, namely a surface immunogenic epitope. The sample magnetic bead mixture should be incubated at room temperature, again for an empirically optimized period. This can be accomplished in a roller bottle, or any other method known to function similarly.

Step II: Use a magnet to aggregate all the beads and wash in buffer to remove non-specific material.

Step III: Add known reagents to the beads that lyse the cells, and their vegetative and resistant forms (spores). The mRNA is prepared by any method also known to those skilled in the art. RNA isolation usually provides a protein denaturation step, and treatment with a chaotropic agent (guanidinium sulfate), which denatures the environmental and cellular ribonucleases present.

Step IV: The sample RNA is hybridized with a capture reverse polarity-triplex forming oligonucleotide (RP-TFO) that is biotinylated and specific for the BCWA mRNA target at pH 5.5. The RP-TFO is specific for a 12-mer polypyrimidine target region with one purine insertion in the target region. See related documents. If the RNA is mRNA, slight heating of the mRNA may aid in triplex formation at target site (reduces secondary on RNA structure). If the RNA is rRNA, more extensive heating (˜90° C.) of the RNA will remove the secondary structure and allow the RP-TFOs to form the stable triplex at the target site. This is the first level of specificity.

Step V: Add any exonuclease that will degrade all non-specific mRNA in a 3′>5′ direction and target mRNA only from the 3′ end to the site of the capture RP-TFO. The capture RP-TFO provides a PNAS, which renders the target nuclease resistant. The enzyme must possess sufficient activity at the pH selected for use, preferably 7.2-7.6 or lower, to allow degradation of non-specific mRNA. At this point, the target/capture probe complex forms the PNAS (protected nucleic acid sequence). The RP-TFO will protect the mRNA target from the RP-TFO binding site to the 5′ capped end of the mRNA target from exonuclease degradation. The reporter probe, as a duplex or triplex, will hybridize to the 5′ end of the target mRNA (between the RP-TFO capture probe and the 5′ end of the mRNA target).

The strategy herein employed requires that the assay pH remain as low as possible to:

Generate the most stable PNAS with the RP-TFO

Prevent environmental ribonuclease assay interference, due to the fact that these possess no activity below pH 7.0. This is the second level of specificity.

Step VI: Streptavidin coated magnetic beads are added to the enzyme treated sample and bind the mRNA target with its attached capture biotinylated RP-TFO, the PNAS.

Step VII: The magnetic beads are washed to remove non-specific material with buffer at pH 7.2.

Step VIII: The mRNA bound magnetic beads are next hybridized with a reporter probe either a duplex forming oligonucleotide or a triplex forming oligonucleotide RP-TFO both possessing an enzyme such as AP. It should be noted that direct mRNA target detection is adequate, in the absence of any signal amplification strategy, due to the rationale that each microbial BCWA would possess thousands of mRNA molecules per derepressed and expressed gene. Later it will be shown that in detection of viral RNA, viral BCWA signal amplification strategies will prove useful for increasing the sensitivity of the overall assay.

Step IX: Wash with buffer to remove unbound reporter probe.

Step X: Resuspend the magnetic bead complex in alkaline phosphatase buffer pH 9.0 which functions to degrade the mRNA and releases the stable reporter probe and attached AP enzyme into the solution phase, and remove the magnetic beads.

Step XI: Add the sensitive 1,2 dioxetane substrates and quantify the light produced.

RNA-TPA for RNA Virus BCWA Detection

RNA-TPA will be presented and will mainly focus on quantification of infectious virions in the large sample being tested for BCWAs. Due to the fact that the direct RNA analysis process has insufficient sensitivity for a good diagnostic process, two different HP signal amplification strategies called CyLite MTRF, presented in related documents, and MACMSA, presented in the following, may be employed. The RNA-TPA process steps are:

Step I: Concentration of viral BCWA in a very large sample.

Again, any sensitive diagnostic assay must process and analyze very large sample volumes as described. Magnetic beads coated with an antibody specific for the viral pathogen to an envelope or other external immunogenic epitope should be added to the sample. The sample/magnetic bead mixture should be incubated at room temperature, again for an empirically optimized period. This can be accomplished in a roller bottle, or any other method known to function similarly.

Step II: Use a magnet to aggregate all the beads and wash in buffer to remove non-specific material.

Step III: Add reagents to the beads to lyse the viral particles. The RNA, usually single stranded, is prepared by any method known to those skilled in the art. RNA isolation usually provides a protein denaturation step, and treatment with a chaotropic agent (guanidinium sulfate), which denatures the environmental and cellular ribonucleases present.

Step IV: The sample RNA is hybridized with a capture RP-TFO that is biotinylated and specific for the viral RNA target at pH 5.5. The RP-TFO is specific for a 12-mer polypyrimidine region on the target with one purine insertion. See related documents. If the RNA possesses secondary structure, slight heating of the RNA may aid in triplex formation at target site. With increasing secondary RNA structure, more extensive heating (˜90° C.) of the RNA will remove the secondary structure and allow the RP-TFOs to form the stable triplex at the target site. This is the first level of specificity.

Step V: Add an exonuclease (3′→5′) to degrade all non-specific ssRNA and target RNA only from the 3′ end to the capture RP-TFO, The capture RP-TFO provides a PNAS, which renders the target nuclease resistant. See related documents. The enzyme must possess sufficient activity at the pH selected for use, 7.2-7.6 or lower, to allow degradation of non-specific (non-target) RNA. The RP-TFO will protect the RNA target from the RP-TFO binding site to the 5′ end of the RNA target from exonuclease degradation. The reporter probe, as a duplex or triplex, will hybridize to the 5′ end of the target RNA (between the RP-TFO capture probe and the 5′ end of the RNA target). The strategy herein employed requires that the assay pH remain as low as possible to:

Generate the most stable PNAS with the RP-TFO

Prevent environmental ribonuclease assay interference, due to the fact that these possess no activity below pH 7.0. This is the second level of specificity.

Step VI: Streptavidin coated magnetic beads are added to the enzyme treated sample and bind the RNA target with its attached capture biotinylated RP-TFO.

Step VII: The magnetic beads are washed to remove non-specific material with buffer at pH 7.2.

Step VIII: The viral RNA bound magnetic beads are next-hybridized with a reporter probe that has any attached immunogenic peptide. In a preferred embodiment, the c-myc peptide is used. See related documents. The reporter probe may be either a duplex forming oligonucleotide, or a triplex forming oligonucleotide (RP-TFO). Direct viral RNA target detection at this point would lack the required sensitivity, requiring the use of any number of HP signal amplification strategies. See related documents. In a preferred embodiment, MACMSA is used to generate an amplified C4a peptide signal in a complement fixation assay previously discussed.

Step IX: The MB/RNA target/reporter c-myc complex is washed to remove unbound reporter probe.

Step X: The magnetic bead complex is placed in a solution of MP RBC stroma, the first stage of the MACMSA process. The MP used in this embodiment is IgG anti c-myc—IgG anti-D used to sensitize Rh POS R2R2 RBCs. The mixture is incubated at room temperature for an empirically determined period. This allows c-myc peptides on target RNA reporter probes to bind anti c-myc on the MP RBC stroma, which in turn fixes and activates complement. Theoretical calculations indicate that 10,000 C4a peptides are produced by each molecule of complement fixed or similarly by every viral RNA particle present

Step XI: The magnetic bead complexes are removed and the supernate assayed for C4a peptides.

Step XII: Perform the magnetic bead C4a sandwich ELISA previously herein presented.

Theoretical-Sensitivities of HP BCWA Diagnostic Processes

A summary of assay sensitivities is provided in Table V. Table V depicts the broad range of biological and chemical warfare agents as they are detected by the MACMSA and RNA-TPA processes. All immunogenic BCWAs can be detected to very low copy numbers equally by either method down to 10-100 BCWA targets.

Table V reflects the sensitivity of the HP complement fixation BCWA diagnostic assays based on quantification both of C4a production as a result of complement fixation and activation in the Classical Pathway and of C3a production as a result of complement activation in the Alternate Pathway. The sensitivity of both approaches to quantification of complement fixation is sufficient for a diagnostic process. Also included is sensitivity using mRNA TPA in bacterial and viral agent assays using alkaline phosphatase.

This invention is further illustrated by the following examples of diagnostic assays employing CMSA and MACMSA, which are not to be construed in any way as imposing limitations upon the scope thereof. On the contrary, it is to be clearly understood that resort may be had to various other embodiments, modifications, and equivalents thereof which, after reading the description herein, may suggest themselves to those skilled in the art without departing from the spirit of the present invention and/or the scope of the appended claims.

MACMSA Process for Environmental Sample Analysis to Detect Pathologic Biological and Chemical Agents

The testing of environmental samples such as water, air, and food for pathologic agents is essential for the health, safety and the economic strength of our society and its citizens. Testing has been historically mandated due to contamination that may be naturally occurring, as a result of industrial chemical leakage and agricultural activities. The strict surveillance of ingested materials has become even more crucial now that the infrastructure of our entire society is at risk of biological/chemical warfare agent (BCWA) attack from terrorist groups. The contamination of environmental sources provides the bio-terrorist with a direct vehicle to rapidly harm larger population segments, reaping panic and resulting in long term economic hardship.

Agencies such as the FDA, EPA, and Department of Agriculture have defined numerous chemical and biological elements in the environment that pose human health concerns and have set up regulations and guidelines to achieve the goal of prompt detection of the harmful agent by continued vigilance. Currently implemented detection processes lack the sensitivity to detect low concentrations of contaminants.

The potential use of BCWA represents a threat by ingestion of low numbers of chemical molecules, due to their genotoxic, i.e., carcinogenic, mutogenic, and teratogenic characteristics, where minimal exposure will exert a rapid, serious pathologic effect on the host.

Natural or industrial carcinogens in the environment present a silent threat due to the lag time for mutational changes to take place and the onset of clinical symptomology. However, limited exposure to BCWA in the environment will produce immediate clinical symptomology often accompanied by rapid death due to the inability to provide medical intervention caused by the inability to detect the BCWA exposure event, and the difficulties encountered in the management of exposure to these agents in the affected host. The BCWA's are selected for use due to their rapid killing ability and the difficulty in medical management of the exposed host.

In light of this danger, a strategy must be adopted that achieves the rapid, accurate, and inexpensive detection of BCWA's that will support immediate quarantine of the contaminated source before large population segments become exposed.

Detection of Pathologic Agents in Environmental Samples

Environmental samples include air, drinking and other water sources, soil, and foodstuffs. A single process that can analyze all environmental sample types for BCWA detection is important for implementing standards and economy.

In the analysis of soil, air, and foodstuffs, the BCWA must be extracted by solution for analysis. The techniques to achieve this are known and practiced, so emphasis will be placed on design of an analysis process that will detect the presence of the pathologic agent in an aqueous sample. For this purpose the example of drinking water analysis will provide the overall model for the analysis of environmental samples.

BCWA Detection in Drinking Water

Drinking water sources are as diverse as the geographic locations where people live. Deep wells, aquifers, rivers, and saltwater desalinization, or combinations thereof provide some water sources. The sources of contamination of municipal drinking water include:

Natural Sources

Algal blooms

Microbial growth; cryptococcus species, and others

Volcanic sources in deep wells

Decay of natural deposits in deep wells

Residential Sources

Septic tank leaching

Chemical contamination of the watershed with oil, gasoline and other chemicals by the general public

Industrial Sources

Chemical plant runoff; plasticizers

Toxic by-product production from the disinfection of water with chlorine and other agents

Agricultural Sources

Crop runoff; pesticides, and fertilizers

Animal waste

Bioterrorist Sources

Biological warfare agents

Chemical warfare agents

Tables VII.1 to VII.5 represent the current EPA National Primary Drinking Water Standards involving the testing of regulated substances.

Table VIII. 1 represents the government unregulated substances currently tested by the city of Albuquerque, N. Mex.

Tables IX.1 to IX.3 represent the Henry I. Stimson Center/Chemical and Biological Weapons Nonproliferation Project current description of biological weapons agents affecting man and anti-plant biological agents.

Current Testing of Municipal Water for Pathologic Agents

In Tables VII, VIII, and IX, a number of pathologic agents are described that require round the clock vigilance in the testing of water sources for the potential threat offered by these agents.

In some cases the detection method involves the collection of contaminants by use of flash evaporation, which offers the best and highest recovery or concentration from a larger sample. Others such as Blue Rayon adsorption and solid phase extraction yield lower recovery rates. Every method currently in use selectively favors certain agents and is limited to the analysis of too small a sample to sensitively detect contaminants. Pesticides, toxins, mutagens and other genotoxic agents must be concentrated in the water sample to determine their presence. Further limitations of concentration methods provide difficulties in securing the appropriate sample for pathologic agent detection.

Current analysis methods to determine the presence of contaminants in the concentrated sample are also diverse and problematic. Of the better processes, spectrophotometric analyses of water is far too expensive and rarely used, leading to the widespread use of insensitive assays such as enzyme immunoassay (EIA), enzyme linked immunosorbent assay (ELISA), and bioassays.

Bioassays are commonly configured for use in nations lacking the financial resources to properly assess the safety and quality of their drinking water sources justifying this measure by the adage something is better than nothing.

Factors Contributing to the Sensitivity of Contamination Detection

The first factor to significantly influence the ability to detect low numbers of toxic molecules is the ability to evaluate a sufficiently large water sample. Mutagenic and genotoxic agents exert their effect on a molecular level and require only minimal exposure to low numbers of molecules to exert their deleterious pathologic effect. It is known that these chemicals have a cumulative effect, namely the continued exposure to low concentrations of toxic substance over a prolonged period will promulgate the disease state.

Secondly, the toxic substance concentrated in the water sample must be sensitively and cost effectively detected. Spectrophotometric analysis is too expensive to be broadly used and the use of relatively insensitive bioassay, EIA, and ELISA techniques do not detect dangerous toxic chemical levels.

HP's Approach to Drinking Water Analysis

HP technologies are a number of diagnostic processes that support analysis of large amounts of sample analyte with the ability to sensitively detect a pathologic target. This is achieved by performing Haystack Processing, which concentrates the pathologic targets in a large amount of sample analyte and then performs signal amplification from potentially very low concentrations of targets present. In the context of water analysis, Haystack Processing would concentrate the low number of pathologic targets in a very large water sample (hundreds to thousands of liters of water can be assayed) in a single analysis.

Furthermore, non-specific target elimination (NTE) is accomplished by use of a selective and sensitive signal amplification process for the detection of concentrated contaminants. The method is called Membrane Assisted Complement Mediated Signal Amplification (MACMSA), which is configured to generate amplified signal exclusively from the pathologic targets, accomplishing both detection and quantification of the number of pathologic targets present.

Specificity is achieved by use of a common characteristic of nearly all pathologic targets (listed in Tables VII, VIII, and IX) namely their immunogenic properties. The pathologic target is concentrated by an anti-target antibody bound to red blood cell membranes, which under the appropriate conditions trigger the generation of an amplified signal for target detection.

MACMSA: A Brief Overview

MACMSA is a complement fixation assay that supports sensitive detection of the pathologic target based on its immunogenic character. The immunogenic target is complexed with sensitized red blood cell (RBC) membranes (stroma). The antibody attached is a molecule pair (MP) possessing two antibodies, the first antibody possesses specificity to the pathologic target and is a complete antibody capable of fixing and activating immune complement and the second antibody attaches the MP to any antigenic site on the RBC, which does not fix or activate immune complement.

Upon complexation of the pathologic target, either a chemical, biological toxin, or virus with the appropriately sensitized MP RBC stroma (Ag/Ab formation) immune complement and cofactors Ca⁺⁺ and Mg⁺⁺ are added, whereupon complement is fixed by the Classical Pathway in an equal molecule amount to the number of pathologic targets present in the sample being tested. The fixation event is dependent upon the binding of the pathologic target to a monoclonal IgG antibody and the presence of another IgG antibody molecule in proximity to fulfill the binding requirements of the Clq molecule, the initial event in complement fixation in the Classical Pathway. The fixation event is followed by complement activation resulting in amplified signal production, namely C4a peptide generation. Theoretically, for each molecule of complement fixed, at least 10,000 C4a peptides are produced. A sensitive sandwich ELISA reaction quantifies these peptides. It is estimated that direct labeling of the C4a peptides with a fluorescent or chemiluminescent label will provide sensitivity to detect down to 100 to 1,000 pathologic target molecules in a large water sample. Other methods used today evaluate much smaller samples and have detection limits in the millions of targets to see a positive assay result (see Table XII).

Upon complexation of a pathologic microbial bacterial and viral targets with the appropriately sensitized MP RBC stroma (Ag/Ab formation) immune complement with Ca⁺⁺ and Mg⁺⁺ cofactors are added, whereupon the Classical and Alternate Complement Pathways are both activated and function to produce amplified signal production, namely C3a peptide generation. Again each bacterial target theoretically generates a minimum of >>100,000 C3a peptides (>>10,000 C3a peptides per viral target) that are quantified by a sensitive sandwich ELISA reaction. It is estimated that direct labeling of the C3a peptide with a fluorescent or chemiluminescent label provides sensitivity of bacterial target detection of 10 to 100 pathologic bacteria in a very large water sample.

Algorithm for Drinking Water Analysis by MACMSA

Analysis for Soluble Chemical Toxins, Genotoxic Agents, and Pertinent Chemical Warfare Agents

Embodiment I: C4a Assay By MB Sandwich ELISA

The MACMSA assay (based on the Classical Complement Pathway) for microcystin-LR toxin in drinking water is presented in the following steps. The toxin results from the natural bloom of blue green algae (cyanobacteria). It is highly hepatotoxic and frequently occurs in natural water blooms around the world. Usual detection of this toxin calls for High Pressure Liquid Chromatography (HPLC) and ELISA assay cost limitations result in assay by a less sensitive bioassay system. Assay of this toxin is representative of all other chemical agents described herein.

Step I: Production of MP RBC Stroma

Human Rh POS (R₂R₂) RBCs are sensitized with the MP composed of two covalently attached antibodies:

MP=IgG anti microcystin toxin—IgG anti-D

IgG anti microcystin toxin confers target specificity to the MP

IgG anti-D confers attachment of the MP to the human Rh POS (R₂R₂) RBC

The sensitized RBCs are next subjected to gentle lysis and the sensitized MP RBC stroma is isolated and washed. The MP RBC is now available for use in the MACMSA toxin assay.

The RBCs that are used may be of any Rh type with appropriate modifications and the blood may originate from outdated units or animal sources, thereby placing no strain on the already inadequate donor blood supply in the world.

Step II: Collection and Concentration of Toxin Molecules

The microcystin MP RBC stroma is placed in a cartridge, which is positioned vertically and possess a fritted disk on each end to permit the antigravity flow of sample water and small non-specific particles. A sufficiently large water sample (many liters) is run through the cartridge at a rate sufficient for attachment of toxin molecules to the MP RBC stroma. In a multiplex test, a cocktail of MP RBC stromas with different chemical target specificities are admixed.

Step III: Wash the Target Loaded MP RBC Stroma

Any buffer at pH 7.0 is used to wash the target loaded MP RBC stroma and remove the buffer.

Step IV: Perform the Complement Fixation Assay

The MP RBC stroma is resuspended in the appropriate amount of complement and Ca⁺⁺ and Mg⁺⁺ cofactors. The cartridge is incubated at room temperature to allow fixation and activation of the classical complement cascade. The complement added may be provided in a lyophilized form to eliminate stringent refrigeration requirements. It is known to those skilled in the art that, complement may be lyophilized and stored at normal room temperature. Once reconstituted, the complement may be stored for up to 12 hours at refrigeration temperatures (4° C.) and still retains sufficient activity upon rehydration.

Step V: Collect the Spent Complement in the MP RBC Stroma Cartridge

Step VI: Perform the Automated C4a Magnetic Bead (MB) Sandwich ELISA

Add MBs coated with an IgG anti C4a monoclonal antibody (C4a capture) and incubate with agitation.

Remove and wash the MB-Mab C4a complex in buffer (pH 7.2)

Add another C4a specific monoclonal antibody that is labeled with an alkaline phosphatase (AP) enzyme to form the structure: MB·Mab C4a·Mab·AP

Wash the magnetic bead

Place MB complex in a solution at pH 9.8 for AP assay using chemiluminescence produced by enzyme reaction with 1,2 dioxetane substrates and incubate to produce chemiluminescence of the substrate.

Remove the MB complex and

Quantify C4a molecules produced and calculate the number of complement molecules fixed based on the number of targets present.

Embodiment II: C4a Assay by Complement Mediated RBC Lysis

The initial assay embodiment steps are identical as described in Embodiment 1 up to the C4a quantification steps.

The following method represents a novel approach to quantify C4a peptide numbers by a rabid, sensitive, cost efficient, and low complexity method. Herein, the spent complement is removed from the water sample laoded MP RBC stroma cartridge and placed into a second cartridge (identical construct) filled with the sensitized intact RBCs. In this embodiment the RBCs are sensitized with the MP: IgG anti C4a—IgG anti-D(Rh). As such complexation of the MP RBC with a single C4a peptide in solution will be sufficient to lyse the RBC in the presence of complement and its cofactors. If sufficient complement units were added to the first analysis cartridge, no additional complement would be needed. The hemoglobin released numbers approximately 10¹¹ molecules per RBC and possesses pseudoperoxidase activity providing the basis for a highly sensitive assay for its detection.

In the fluorine blue assay for hemoglobin detection and quantification, a compound 2-7 diaminofluorene when exposed to a hemoglobin molecule forms fluorine blue which is detectable with a colorimeter at wavelength 610 nm. The assay is documented to possess more sensitivity than Hb release and absorption measure at 410 nm and even 51 Cr loading of intact RBCs and label detection in the solution phase upon RBC lysis.

The high sensitivity results from the production of much greater than 100 billion (>>10¹¹) fluorine blue molecules per lysis of a single RBC, which in an excess of MP RBCs can represent lysis by a single C4a peptide.

Analysis for Bacterial Particles from Water Pollution and Bacterial Biological Warfare Agents

Embodiment I: C3a Peptide Assay By Magnetic Bead Sandwich ELISA

The MACMSA assay (based on the Alternate Complement Pathway) for bacteria present in a drinking water sample is presented for the enterotoxigenic strains of E. coli. Presence of these strains may be the result of pollution or terrorist activity. The process that follows is similar for all bacterial species with minor modifications.

Step I: Production of MP RBC Stroma

Human Rh POS (R₂R₂) RBCs are sensitized with the MP composed of two covalently attached antibodies:

MP=IgG anti E. coli toxigenic surface protein—IgG anti-D

IgG anti E. coli toxigenic surface protein confers target specificity to the MP

IgG anti-D confers attachment of the MP to the human Rh POS (R₂R₂) RBC

The sensitized RBCs are next subjected to gentle lysis and the sensitized MP RBC stroma is isolated and washed. The MP RBC is now available for use in the MACMSA toxin assay.

The RBCs that are used may be of any Rh type with appropriate modifications and the blood may originate from outdated units or animal sources.

Step II: Collection and Concentration of E. coli (Toxigenic) Bacterium

The E. coli (toxigenic) MP RBC stroma is placed in a cartridge, which is positioned vertically and possess a fritted disk on each end to permit the antigravity flow of the water sample. A sufficiently large water sample (many liters) is run through the cartridge at a rate sufficient for attachment of bacterial particles to the MP RBC stroma. In a multiplex test, a cocktail of MP RBC stromas with different bacterial target specificities are admixed.

Step III: Wash the Target Loaded MP RBC Stroma

Any buffer at pH 7.0 is used to wash the target loaded MP RBC stroma and remove the buffer.

Step IV: Perform the Complement Fixation Assay

The MP RBC stroma is resuspended in the appropriate amount of complement along with a Ca⁺⁺ and Mg⁺⁺ cofactor. The purpose of Mg⁺⁺ addition is to drive the activation of the Alternate Complement Pathway optimal for bacterial activation of complement and amplified C3a peptide production. The cartridge is incubated at room temperature to allow activation of the Alternate Pathway and subsequent C3a peptide production.

Activation of the Alternate Pathway for this target, theoretically, results in more extensive production of C3a peptides in numbers >>100,000 per bacterial target cell as known to those skilled in the art. The complement added may be provided in a lyophilized form.

Step V: Collect the Spent Complement in the MP RBC Stroma Cartridge

Step VI: Perform the Automated C3a Magnetic Bead (MB) Sandwich ELISA

Add MB coated with an IgG anti C3a monoclonal antibody (C3a capture) and incubate with agitation.

Remove the MBs and wash the MB-Mab C3a complex in buffer (pH 7.2)

Add another C3a specific monoclonal antibody that is labeled with an alkaline phosphatase (AP) enzyme to form the structure: MB-Mab C3a·Mab·AP

Wash the magnetic bead

Place MB complex in a solution at pH 9.8 for AP assay using chemiluminescence produced by enzyme reaction with 1,2 dioxetane substrates as previously described.

Quantify C3a molecules produced and calculate the number of complement molecules fixed based on the number of targets present.

EMBODIMENT II: C3a Assay by Complement Mediated RBC Lysis

The initial assay embodiment steps are identical as described in embodiment I up to the C3a quantification steps. The C3a assay may also be achieved by use of sensitized RBC lysis where the sensitized RBCs are MP RBC: MP=IgG anti C3a-IgG anti-D (Rh). Again, complexation of a single C3a peptide with the intact MP RBCs in the presence of complement and cofactors will result in MP RBC lysis and release of 10¹¹ hemoglobin (Hb) molecules per MP RBC. The fluorine blue assay for Hb has been previously described in this document.

Analysis for Viral Particles from Water Pollution and Viral Biological Warfare Agents

Embodiment I: C4a Peptide Quantification by Magnetic Bead Sandwich ELISA Assay

The MACMSA assay (based on the Classical Complement Pathway) for smallpox virus (variola major) detection in drinking water is presented in the following steps. Presence of these viruses may be a result of terrorist activity. The process that follows is similar for all viral species with minor modifications.

Step I: Production of MP RBC Stroma

Human Rh POS (R₂R₂) RBCs are sensitized with the MP composed of two covalently attached antibodies:

MP=IgG anti smallpox coat protein—IgG anti-D

IgG anti smallpox coat protein confers target specificity to the MP

IgG anti-D confers attachment of the MP to the human Rh POS (R₂R₂) RBC

The sensitized RBCs are next subjected to gentle lysis and the sensitized MP RBC stroma is isolated and washed. The MP RBC is now available for use in the MACMSA virus assay.

The RBCs that are used may be of any Rh type with appropriate modifications and the blood may originate from outdated units or animal sources.

Step II: Collection and Concentration of Viral Particles

The smallpox MP RBC stroma is placed in a cartridge, which is positioned vertically and possess a fritted disk on each end to permit the antigravity flow of sample water. A sufficiently large water sample (many liters) is run through the cartridge at a rate sufficient for attachment of viral particles to the MP RBC stroma. In a multiplex test, a cocktail of MP RBC stromas with different viral target specificities are admixed.

Step III: Wash the Target Loaded MP RBC Stroma

Any buffer at pH 7.0 is used to wash the target loaded MP RBC stroma and remove buffer.

Step IV: Perform the Complement Fixation Assay

The MP RBC stroma is resuspended in the appropriate amount of complement and Ca⁺⁺ and Mg⁺⁺ cofactors. The cartridge is incubated at room temperature to allow fixation and activation of the complement cascade. The complement added may be provided in a lyophilized form.

Step V: Collect the spent complement in the MP RBC stroma cartridge

Step VI: Perform the automated C4a magnetic bead (MB) sandwich ELISA

Add MB coated with an IgG anti C4a monoclonal antibody (C4a capture) and incubate with agitation.

Remove and wash the MB-Mab C4a complex in buffer (pH 7.2)

Add another C4a specific monoclonal antibody that is labeled with an alkaline phosphatase (AP) enzyme to form the structure: MB·Mab C4a·Mab·AP

Wash the magnetic bead complex

Place the MB complex in a solution at pH 9.8 for AP assay using chemiluminescence produced by enzyme reaction with 1,2 dioxetane substrates.

Quantify C4a molecules produced and calculate the number of complement molecules fixed based on the number of targets present. A minimum of 10,000 C4a peptides is expected for the presence of a single viral particle.

Embodiment II: C4a Quantification by Complement Mediated Sensitized MP RBC Intact Cell Lysis

The initial assay embodiment steps are identical as described in embodiment 1 up to the C4a quantification steps. The C4a assay may also be achieved by use of sensitized RBC lysis where the sensitized RBCs are MP RBC: MP=IgG anti C4a-IgG anti-D (Rh). Again, complexation of a single C4a peptide with the intact MP RBCs in the presence of complement and cofactors will result in MP RBC lysis and release of 10¹¹ hemoglobin (Hb) molecules per MP RBC. The fluorine blue assay for Hb has been previously described in this document.

Theoretical Sensitivity of MACMSA in Drinking Water Analysis of Large Water Samples

The following chart indicates the theoretical sensitivity limits of detection of the following targets:

| | | | Number Of | | |
|---|---|---|---|---|---|
| | | | Targets Detectable | | |
| | | | In A Large Water Sample* | | |
| | Signals Produced Per | | Assay Efficiency | | |
| Target | Target | | 100% | — | 10% |
| Any immunogenic | C4a | 10,000 | 100 | molecules | 1000 |
| chemical | | | | | |
| Biologic toxin | C4a | 10,000 | 100 | molecules | 1000 |
| Bacterial particle | C3a | 100,000 | 1 | bacterial | 10 |
| | | | | particles | |
| Viral particle | C4a | 10,000 | 100 | viral | 1000 |
| | | | | particles | |

*C3a analysis by a sensitive sandwich ELISA assay

Toxin Detection Example

Use of MACMSA Analysis for the Ultrasensitive Detection of Aflatoxin B1 in Tobacco Processates

Use of the SLESA embodiments referred to as CMSA and MACMSA can be demonstrated for the ultra-sensitive detection of Aspergillus and the mycotoxins aflatoxin (AFB1). Aflatoxins are highly toxic and carcinogenic factors produced by mold contamination of soil-contacted foodstuffs such as peanuts and tobacco. They are usually produced by Aspergillus flavus and Aspergillus parasiticus and have been characterized as highly unsaturated molecules with a coumarin nucleus.

Aflatoxin B1 and G1 are the parent compounds and are potent carcinogens and have been shown to exert their carcinogenic effect by interaction with cellular nucleic acids (via adduct formation and base change). Aflatoxin B1 has been shown to suppress DNA, RNA and protein synthesis in rat liver cells. These mycotoxins, upon activation have been also shown to mutate both the p53 tumor suppressor gene as well as the K-ras genes. These mutations (guanine and cytosine transitions) implicate these mycotoxins as the causal agent in many human cancers, such as breast, colon, lung, pancreatic and others.

The mechanism of aflatoxin B1 reaction is through the formation of DNA adducts supported by the active mode of transport of extracellular toxin into eukaryotic cells, probably mediated by its lipid-nature. Similarly, liposomes themselves, lipoid in nature, are afforded rapid uptake through the cell membrane.

Processes and strategies are continually being developed that will reduce the amount of aflatoxin in the consumed product; however, the inability to sensitively detect very low levels of mycotoxin prove the limiting factor in attempts to improve the safety for use of the ingested foodstuff.

Currently, assays for AFB1 are accomplished by chromatography, including high-pressure liquid chromatography (HPLC), reversed-phase liquid chromatography, thin-layer chromatography, adsorption chromatography, immunoaffinity chromatography, gas chromatography; enzyme-linked immunoadsorbent assay (ELISA), fluorescent immunoassay, radioimmunoassay; spectroscopy, including mass spectroscopy, infrared spectroscopy, raman spectroscopy, packed-cell fluorescent spectroscopy; polymerase chain reaction (PCR), supercritical fluid extraction, bio-luminescence, chemical luminescence, and combinations thereof. Fluorescent immunoassay is a presently preferred best mode for assaying for aflatoxin on tobacco with a lower limit of sensitivity of parts per billion (trillions of molecules remain undetectable in the final processed material).

All of these above diagnostic detection techniques lack sensitivity leading to the generation of false negative diagnostic results. These assays currently offer sensitivities no less than parts per billions, meaning that even at the lowest detection level of these toxins very high numbers of molecules still remain present to achieve DNA adduct status in the tobacco user and pre-dispose him/her to a number of cancers.

The aflatoxin B1 presence in tobacco provides a major health risk for users that have been recognized. Attempts have been made to reduce and limit its presence and have been met with strong criticism due to the inability to determine its presence with high sensitivity.

Currently, FDA does not regulate AFB 1 levels but does place limits of mold infection of raw tobacco to 300 parts per billion. With the knowledge that production of a single guanine or cytosine transition can predispose an individual to cancer, due to a germ cell mutation, the burden is upon diagnostics to sensitively detect the presence of aflatoxin B1 at much lower levels than is currently attainable. This increased sensitivity coupled with any effective tobacco treatment process to eliminate aflatoxin B1 can result in production of a tobacco product with much reduced risk of cancer production, a “safe” tobacco.

A technique, discussed herein, called Membrane Associated Complement Mediated Signal Amplification (MACMSA) has been developed for the detection of soluble proteins, lipids, polysaccharides, and lipopolysaccharides in solution. The method relies upon the presence of an antigenic epitope on the molecule and a monoclonal antibody specific to this epitope, both currently available for the AFBI molecule. This interaction (antigen/antibody complex) will fix and permit complement activation, and the activation will be amplified by the presence of a lipid substrate, in this case, the sensitized RBC stroma. Again as described, complement fixation and activation will be monitored by C3a peptide production and its quantification, also herein described. This involves the classical complement fixation pathway.

Similarly, the presence of Aspergillus species organism producing the AFBI toxin can be detected present in very low copy numbers in tobacco early in its processing. This is accomplished through Complement Mediated Signal Amplification (CMSA) and involves the alternate complement fixation pathway, namely the interaction of the molds cell surface polysaccharides and lipopoly-saccharides with complement Factor B, Factor D, and properdin. No antibody is necessary and no complement fixation occurs, but again complement activation occurs and can be monitored by C3a peptide production and its quantification, also herein described.

Utilizing CMSA and MACMSA, one can configure ultra-sensitive diagnostic tests to follow the tobacco from its start through each stage of its processing and resulting in the production of a tobacco/end product that is essentially devoid of AFB1. Table VI presents a detection scheme for Aspergillus species assay and soluble AFB 1 assay during the tobacco processing steps.

The following are the steps that comprise the quantitative assay for the organism that is present that produces the toxin. Any toxin producing organism known can be similarly detected.

Quantitative and Automated Raw Tobacco Assay for Aspergillus Species Organisms: C3a Sandwich Elisa

Step I: Prepare batch homogenate for testing in buffer in a microtiter plate well.

Step II: In one embodiment, add magnetic beads to the well coated with a material specific for fungal cell walls, as opposed to other microbes (differential binding of intact fungi) and mix and incubate for optimum time and temperature. In another embodiment this may be an antifungal antibody fragment devoid of Fe fragment.

Step III: Remove the beads, wash, and place in a new plate well.

Step IV: Add fresh complement and cofactors and mix.

Step V: Incubate at room temperature for an optimized time.

Step VI: Remove the magnetic beads and place the supemate in a new well, to assay for C3a peptides generated, containing magnetic beads coated with the IgG anti C3a capture monoclonal antibody.

Step VII: Wash the magnetic beads and place them in a new plate well.

Step VIII: Add to the well IgG anti C3a reporter monoclonal antibody conjugated with an enzyme such as alkaline phosphatase and mix.

Step IX: Wash the magnetic beads to remove unbound enzyme and place the beads into a new plate well.

Step X: Add the 1,2 dioxetane chemiluminescent substrate and incubate at optimal time and temperature.

Step XI: Quantify the light produced sensitive down to subattomale numbers of enzyme molecules (1,000 to 10,000).

The following are the steps that comprise the ultra-sensitive assay for the presence of the soluble AFB1 aflatoxin.

It is important to herein note that any toxin or carcinogen known can be similarly assayed such as the most widely studied and suspected environmental carcinogens in lung cancer: polycyclic aromatic hydrocarbons (PAHs) including benzo(a)pyrene (BzP) and 4-(methylnitrosoamino)-1-(3-pyridyl)-1-butanone (NNK), along with the AFB1, by a similar method. Similar is the case for use in BCWA detection.

Interestingly, all these and other carcinogens and teratogens form adducts with specific DNA bases, a major factor exploited to allow its sensitive extraction and isolation from solution in vitro. Furthermore, all the above hydrocarbons are proven to cause specific mutations to the p53 tumor suppressor and K-ras genes.

Quantitative and Automated Tobacco Processing Assay for Soluble AFB 1: Capture Strategy One-DNA Adduct Formation

Step I: Prepare a batch homogenate for testing of the presence of AFB1 in buffer and place in a microtiter plate well.

Step II: Add magnetic beads coated with poly G•poly C duplex DNA (stable duplex) to allow adduct formation by soluble AFB1 molecules in the supemate of a sample of the tobacco solution processate.

Step III: Incubate at conditions favorable to formation of the adduct to bind soluble AFB1 present to the GC duplex on the magnetic beads.

Step IV: Wash the magnetic beads and place them in a new plate well.

Step V: Add sensitized RBC stroma (sensitized with antibody pair: IgG anti-D-IgG anti AFB1).

Step VI: Incubate at conditions favorable to formation of the AFB1 adduct/anti AFB1 red blood cell membrane complex (AFB1—MP RBC).

Step VII: To the same plate well add fresh complement and cofactors and incubate at room temperature to allow production of C4a peptides.

Step VIII: Remove the magnetic beads and transfer the remaining supernate to another plate well containing magnetic beads coated with IgG anti C4a capture monoclonal antibody and mix.

Step IX: Remove the magnetic beads and wash to remove non-specific material, and transfer to another plate well.

Step X: To the well, add IgG anti C4a reporter monoclonal antibody conjugated with AP, mix and incubate an optimal time.

Step XI: Wash the magnetic beads to remove non-specific enzyme and

Step XII: Add the 1,2 dioxetane chemiluminescent substrate to the well and incubate at an optimum time and temperature.

Step XIII: Quantify the light produced and confirm target presence, sensitive down to sub attomole amounts of enzyme.

Quantitative and Automated Tobacco Processing Assay for Soluble AFB1: Capture Strategy Two—Affinity Molecule Association

Step I: Place batch homogenate for testing in a microtiter plate well.

Step II: Add magnetic beads to the well coated with a material (lipophilic or other) that binds to AFB1 or other lipid toxins.

Step III: Remove magnetic beads, wash to get rid of non-specific material, and place beads in another plate well.

Step IV: Add sensitized RBC stroma (sensitized with antibody pair-IgG anti-D—IgG anti AFB1) to the well.

Step V: Incubate at conditions favorable to formation of the AFB1/anti AFB1 complex, optimal time and temperature.

Step VI: Add fresh complement to the well and incubate at room temperature to allow production of C4a peptides

Step VII: The magnetic beads are removed and the supernate is placed in another plate well to which is added magnetic beads, coated with IgG anti C4a capture monoclonal antibody, to capture C4a produced and mix.

Step VIII: Wash the magnetic beads to remove non-specific material and place the beads in a new plate well.

Step IX: Add IgG anti C4a reporter monoclonal antibody conjugated with AP to the beads and mix.

Step X: Wash the magnetic beads to remove unbound conjugate and place in a new plate well.

Step XI: Add the 1,2 dioxetane chemiluminescent substrate to the well and incubate at optimal time and temperature to generate light.

Step XII: Quantify the visual light produced denoting target or AFB1 presence, sensitive down to sub attomole amounts of enzyme.

Quantitative and Automated Tobacco Processing Assay for Soluble AFB1: Capture and Assay Strategy Three—Sensitized RBC Lysis (Sensitized with the Ab pair IgG ANTI-D—IgG anti AFB 1)

Step I: Place batch homogenate for testing in microtiter plate well.

Step II: Remove particulate material by filtration (passive) through a membrane, gravity driven.

Step III: Add RBC sensitized cells (anti IgG anti-D—IgG anti AFB 1) to clear filtrate and add fresh complement.

Step IV: Monitor RBC lysis spectrophotometrically.

This assay may be of value in the early processing steps where AFB1 molecules range in the multiple trillions.

In this example any of HP signal amplification technologies can be fully functionally substituted for the CMSA and MACMSA process in terms of generation of target signal. Any technology that functions to similarly generate this highly important signal can be interchanged for assays in all sample areas.

This illustrates the ability of the interchangeable use of these interactive processes and embodiments.

Blood Safety Example

Use of MACMSA Analysis for the Detection of Bacterial Contamination in Platelets

Blajchman (2000) reviewed transfusion associated septic reactions during or after transfusion of cellular blood components and found that the presence of bacteria in cellular blood products has been a problem for many decades and currently is the most common microbiological cause of transfusion-associated morbidity and mortality. He noted that these transfusion-associated septic reactions are more prevalent due to contaminated platelet concentrates than those due to red cell concentrates. He concluded that the prevalence of contaminated cellular blood products is 1 in 2,000, wherein not all are sufficiently contaminated to cause morbidity and mortality of the recipient. He estimates that the prevalence of transfusion-associated sepsis is 1 to 50,000 for platelet units and 1 to 500,000 for red blood cell units. What is necessary is a method of assessing the state of sterility of the platelet unit.

Platelet Collection Procedures

In the United States, the FDA regulates blood-banking activities. The approved platelet production regimen requires, one, blood collection in anticoagulant solution ACD (acid citrate dextrose) in one of a number of connected bags, two, low speed centrifugation of the bags thereby separating the white blood cells, the red blood cells and the plasma, three, high speed centrifugation separation of the platelets in the plasma, and reconstituting the platelets in approximately 50 to 60 milliliters of the plasma. The entire system is closed (attached sterilized bags) and each blood fraction is isolated in a separate bag.

The platelet fraction must be incubated no longer than 5 days with rocking on a moving platform to keep the platelets disaggregated. The pH is stable for the 5-day period of incubation at 20° C. to 24° C.

Problems often arise when, during the phlebotomy process, bacterial contamination is introduced into the platelet fraction, which with aeration, rocking, and high incubation temperatures (20° C. to 24° C.), begins logarithmic bacterial growth. When undetected, the resulting bacteria may cause the recipient of the platelet unit to develop a systemic bacteremia, often a life-threatening situation.

Testing of the Platelet Unit Prior to Administration to Patient

The unit of platelets stored under conditions optimal for bacterial growth must be assayed before usage to insure the sterility of the product. The best test result could be obtained by separation and analysis of the entire platelet fluid volume (50 to 60 milliliters) of plasma, while replacing the plasma with a suitable sterile buffer. This large sample would support the highest sensitivity (no false negatives) of the assay to assure platelet sterility before administration.

The challenges involved in platelet sterility testing are numerous and range from:

Adequate plasma processing for optimal collection of the bacterial contaminants,

Reduction of the plasma volume to concentrate the bacterial contaminants,

Treatment of the concentrated bacterial contaminants to generate an amplified signal to detect its presence, even at ultra-low numbers,

Quantification of the signal to determine the extent of bacterial contamination present.

Furthermore, the assay must possess the highest levels of specificity and sensitivity. In previous documents, the specificity of an assay could be assured by following the edict of non-specific target elimination (NTE). NTE functions by use of a Haystack Processing technology such as Target Protection Assay (TPA) on a molecular level (DNA/RNA), or Complement Mediated Signal Amplification (CMSA) on a cellular or soluble protein/chemical level.

TPA functions by reducing the background signal by use of enzymes to destroy non-specific analyte that are unable to destroy the protected target molecules. In CMSA, an amplified signal is generated by complexation of a cell subset with a monoclonal antibody specific for it, which in the presence of immune complement reagent and its cofactors will fix and activate complement. The activation process results in amplified numbers of cascade activation products such as C3a, C4a, C5a, etc. The detection of these amplified products is used to detect the presence of low numbers of cells present from the specific subset of interest.

No interference exists from production of these amplified products from the normal cell population. Only the presence of antibody/antigen complexes can fix and activate immune complement. Normal cells do not, alone, activate complement. Thus NTE is achieved.

Membrane Assisted Complement Mediated Signal Amplification (MACMSA) was developed to support NTE in the detection of soluble protein and other immunogenic chemical molecules. Herein, a soluble immunogen interacts with a monoclonal antibody sensitized red blood cell membrane. The antibody is specific for the immunogen resulting in Ag/Mab RBC membrane complexation and subsequent fixation and activation of complement. This activation causes production of the amplified cascade proteins previously discussed as signals.

Sensitivity of the diagnostic assay for bacterial contamination in platelets is assured by analysis of a large amount of sample analyte for the presence of the bacterial contaminant. In this assay, analysis of the entire 50 to 60 cc plasma volume in the platelet unit would yield a highly sensitive result as to the sterility and safety of the platelet unit.

Molecular Level Detection of Bacterial Contaniination in Platelet Units

The potentially large number of bacterial contaminants present poses difficulties on a molecular level to find DNA, mRNA, rRNA, and tRNA sequences that are shared by all. Furthermore, the rRNA and tRNA possess significant secondary and tertiary structure, which would preclude probe hybridization analysis processes. DNA and mRNA analysis schemes are possible, however, the analysis of 50 to 60 milliliter volumes of a supposed sterile plasma sample on a molecular level is complicated and may not provide the assay sensitivity necessary.

The Approach to Detection of Bacterial Contamination in Platelet Units

One approach is to level the playing field in platelet contamination diagnostics by selection of a characteristic common to all bacterial organisms, as a basis for assay process design. This would allow a common analysis process to be designed that can detect the wide range of bacterial agents required. Most bacteria fall into two categories based on the chemical characteristics and structure of their cell walls. These are referred to as Gram-positive (some bacillus, streptococcus and staphylococcus species) and Gram-negative (coliform, salmonella, shigella, and other enterobacter species).

It is know that Gram-negative bacteria incubated in normal human serum release complexes that contain three conserved Gram-negative bacterial membrane proteins called OMPs and bacterial lipopolysaccharide called LPS. OMP is composed of outer membrane protein A (OMP A), peptidoglycan-associated lipoprotein (PAL), and murein lipoprotein (MLP). OMPA, PAL, and MLP are released and circulate in Gram-negative sepsis and it is known that a portion of the released OMPs are tightly associated with LPS (Hellman, 2001).

Gram-positive bacteria possess a cell wall composed of a peptidoglycan macromolecule with attached accessory molecules such as teichoic acids, teichuronic acids, polyphosphates, or carbohydrates. It is also assumed that peptidoglycan (PG) molecules are also released in the growth medium (plasma) upon incubation similar to the phenomenon demonstrated in Gram-negative bacteria.

The presence of immunogenic peptidoglycan in both Gram-positive and Gram-negative microorganisms and culture supernates provides an opportunity to detect their presence in the platelet unit that exploits the immunogenicity of peptidoglycan. Any other immunogenic protein common to either Gram-positive or Gram-negative bacteria or both may be exploited similarly.

A novel method to sensitively detect the presence of bacterial contamination in platelet units will now be presented. The assay process is called MACMSA as previously described. This involves passage of the plasma in the platelet unit through a cartridge containing sensitized red blood cell (RBC) membrane or stroma. The stroma in one embodiment of the assay is Rh POS (R₂R₂) RBC membranes that were sensitized by the following molecule pair (MP):

IgG anti PG−IgG anti-D (Rh)

Mab #1 attached to Mab #2 where Mab #1 is specific for peptidoglycan, which is present in the cell walls of both Gram-positive and Gram-negative organisms. It has also been show to be secreted from Gram-negative bacteria incubated in normal human serum (Hellman, 2001) at many fold excess over the number of bacteria themselves. The antibody would have affinity for bacterial cells and soluble peptidoglycan moieties.

Mab #2 possesses specificity for the D (Rh) site on the Rh POS RBC. This antibody is required to sensitize the RBC (Rh POS) without fixing complement, a phenomenon known to those skilled in the art.

The cartridge volume is directly related to the volume of diluent assayed. For this application where ˜50 milliliters of plasma will be passed through the cartridge, a 10 cc volume cartridge would be appropriate. This cartridge will contain 5 milliliters of packed sensitized RBC stroma. The column, filled with stroma, possesses a large porosity membrane or fritted disk on both ends that will retain the sensitized RBC stroma as the plasma is passed through the cartridge. To avoid gravity and plasma flow pressure packing of the stroma, the diluent is fed in an antigravity manner (vertical oriented column with inflow of plasma into the bottom). The flow rate must be empirically determined, however, a typical rate should range from 1 milliliter to 2 milliliters per minute (30-60 minutes) cartridge loading time. All aspects of cartridge design and operation parameters must assure binding of all bacterial contamination targets to the stroma.

The choice of RBC membrane as a capture matrix was not accidental. It is known that antigen/antibody (Ag/AB) interactions fix immune complement under certain conditions. It is also known that this Ag/AB complex, where the antigen is affixed to a RBC or RBC membrane, in proximity to any lipid membrane will support efficient fixation and greatest activation of complement possible.

In a novel manner, MACMSA reverses the situation wherein the antibody with peptidoglycan specificity is attached to the RBC membrane. Thus, complexation of the bacteria (Gram-positive and Gram-negative) and soluble peptidoglycan moieties with the appropriate MP (IgG anti PG) RBC stroma in the presence of immune complement reagent and its required cofactors will allow fixation and maximal activation of the immune complement cascade.

In this invention, the complement is activated via the Classical Complement Pathway requiring Ca++ as a cofactor producing C4a peptides in abundance. Another pathway present is the Alternate Complement Pathway, which requires Mg++ and activated complement via a different. Activation of this pathway produces even more abundant numbers of the C3a peptide. This is represented in the following:

| | | Amplified Signal | |
|---|---|---|---|
| | Complement | Produced And | |
| Target | Cascade Activation | Theoretical Number | |
| Gram (+) intact bacteria | Alternate Pathway | >>100,000 | C3a |
| Gram (−) intact bacteria | Alternate Pathway | >>100,000 | C3a |
| Soluble PG (from both above) | Classical Pathway | 10,000 | C4a |

Generation of an Amplified Signal by the Presence of Captured Peptidoglycan Targets

As previously stated, the peptidoglycan targets are concentrated by passage of the plasma solution through the stroma cartridge and by attachment of the PG targets to the appropriately (IgG anti-D) sensitized MP RBC membranes. The PG target stromal complex in 10 milliliters of water or buffer is replaced and stroma resuspended in the following solution:

Immune complement,

Ca⁺⁺ and Mg⁺⁺ pH ˜7.2.

The complement filled PG target loaded stroma cartridge is incubated at room temperature to permit the fixation and activation of the complement cascade. This results in the generation of several different complement cascade activation products at significantly amplified levels. In one MACMSA embodiment, the C4a peptide is theoretically produced at a ratio of 10,000:1 [C4a:PG target]. In other assay embodiments, any other complement activation product may be used as a signal; however, none are amplified to the extent of the C4a peptide by the Classical Pathway. Table I presents some of the possible complement activation product signals. Each activation product is analyzed by sandwich ELISA after labeling with Alkaline Phosphatase (AP) and reaction with sensitive chemiluminescent substrates to detect and quantify the activation products present. As depicted in Table I, detection of the C4a and C3a peptide products produced, theoretically, supports single PG and bacterial target detection in the 50 milliliter to 60 milliliter plasma volume in the platelet unit.

It must be restated that the PG targets include:

Gram-positive bacterial particles

Gram-negative bacterial particles

PG molecules released from each of the above during growth in the plasma in the platelet unit. This soluble PG target will further help to signal amplify the presence of bacteria growing in the platelet unit.

For these reasons, the choice of the PG target to monitor platelet units for bacterial contamination should result in a highly sensitive assay.

MACMSA Platelet Contamination Assay Characteristics

The basics of the assay have been herein, presented. The assay can be fully automated or configured as a semi-automated assay. The total assay time will range from 2.0 to 3.0 hours.

The assay will detect most bacteria with the requirement for a high affinity and high avidity Mab with specificity for PG, which does exist and is currently available. The assay will detect bacterial contaminants that are alive or dead. Depending on the nature of the platelet unit contamination and the plasma source, it may be assumed that the majority of the bacteria are live.

The molecule for target detection presented herein is only representative. Any molecule common to both Gram-positive and Gram-negative bacteria or combinations of different molecules from both can be used as targets in the MACMSA assay.

The MACMSA Bacterial Contamination Assay Used in Blood Platelet Testing

The process for MACMSA analysis is presented as follows:

Step I: Collect the plasma (˜50 to 60 milliliters) from the platelet unit and replace with an appropriate buffer.

Step II: Pass the total plasma volume a 10-milliliter cartridge filled with 5 milliliters of packed red blood cells that are sensitized with the molecule pair:

IgG anti PG—IgG anti-D

The parameters of this operation have been presented. All the PG targets previously discussed selectively bind to the sensitized RBC stroma.

Step III: The cartridge is washed in buffer to remove non-specific material.

Step IV: Complement and cofactors (Ca⁺⁺ and Mg⁺⁺) are added to the cartridge and the flow stopped. The cartridge filled with complement is incubated for 15 to 30 minutes at room temperature under conditions to fix and activate complement by any of the pathologic targets present in the cartridge on the MP RBC stroma.

Step V: Run buffer through the cartridge and collect the void volume effluent (namely all the complement filling the cartridge) containing all C4a peptides generated by pathologic target presence and activation of the Classical Complement Pathway. A similar strategy is used for C3a peptides produced by intact bacterial activation of the Alternate Pathway, but will not be discussed.

Step VI: Add magnetic beads to the effluent coated with the capture antibody IgG anti C4a and incubate at room temperature (perform the C4a sandwich ELISA).

Step VII: Using a magnet, collect the MB IgG anti C4a/C4a complexes and Resuspend in a small volume of buffer.

Step VIII: Wash to remove non-specific unbound material.

Step IX: Add reporter IgG anti C4a·AP (conjugated with alkaline phosphatase-AP)

Step X: Wash to remove non-specific unbound material.

Step XI: Collect the complexes MB IgG anti C4a/C4a/IgG anti C4a·AP with a magnet and resuspend in alkaline phosphatase buffer pH 9.0.

Step XII: Add the sensitive chemiluminescent substrate (1,2 dioxetane) sensitive down to sub-attomole amounts of enzyme.

Step XIII: Quantify C4a production, which is an indicator of the extent of complement fixation, and an indicator of number of pathologic target present in the plasma sample.

Food (also Water) Safety Example use of MACMSA Analysis for the Detection of Bacterial Contamination in Foodstuffs

The challenges involved in testing foods for contamination are numerous and range from:

Adequate food processing for optimal collection of the contamination,

Reduction of the sample volume to concentrate the contamination,

Treatment of the concentrated contamination to generate an amplified signal to detect its presence, even at ultra low numbers,

Quantification of the signal, to determine the extent of presence of the contamination.

Further complicating the design of a food testing system is the necessity to detect a wide range of microbial and chemical contaminants. A brief listing of these agents of biological and chemical warfare importance can be found on the CDC web site http://www.bt.cdc.gov/Agent/Agentlist.asp.

If this wasn't demanding enough, a food testing diagnostic process must also possess the highest levels of specificity (no false positives) and sensitivity (no false negatives). Further included would be the requirements for low cost, speed of process analysis, and the ability to automate the process.

The present invention levels the playing field in food safety testing by selecting a characteristic common to all microbial and chemical contamination agents as a basis for process design. This would allow a common analysis process to be designed that can detect the wide range of microbial and chemical agents required.

Each organism and chemical moiety on the planet possesses unique antigenic properties, which can provide its singular detection. Surface antigenic markers on the cell wall, cell membrane, and envelope of microbes as well as antigenic epitopes on all chemical species can be used to produce monoclonal antibodies (Mabs) specific for the unique antigenic marker. The specific Mabs are selected by their high avidity and affinity to the unique antigenic marker.

Monoclonal antibodies are currently available for microbes and chemicals in general, due to their development for use in taxonomy, serotyping, therapeutics, and diagnostics (usually ELISA or Enzyme Linked Immunosorbent Assay). Technology for production of Mabs is plentiful and time and costs are reasonable. Remember, once a clone is isolated it can be used forever.

The challenges in food testing process design will now be discussed.

Collection of the Contamination

The food to be analyzed must be treated to separate the food material from the microbial or chemical contaminant. One way this may be accomplished could involve the liquifaction of the solid foodstuff by blending and dissolution in a large volume of water (roughly 1:10 ratio of volume of solid foodstuff to diluent). The complete liquifaction will encourage the microbe or chemical to enter the liquid phase for separation from the solid phase to facilitate collection of the contaminant. Any direct analysis of the solid food could only assay miniscule (microgram to nanogram) quantities of the foodstuff.

In order to insure the highest sensitivity of the assay, a sufficiently large mass of the foodstuff must be tested. In this assay process design, size of foodstuff sample is not limited and the larger the sample, the better the assay sensitivity approaching 100%. This concept is referred to as Haystack Processing, wherein the entire haystack is tested for the presence of the elusive needle (contaminant), not just a pinch of hay, which small sample would result in maximal sampling error in the foodstuff analysis result.

The liquid phase must now be separated from the particulate material in the homogenized sample. This may be accomplished by centrifugation or filtration; the former may provide better chemical contaminant isolation, while the latter may provide better microbial contaminant isolation. This must be empirically determined.

At this point, a large water sample from a source suspected as containing contamination can be introduced. The following process is identical for testing either sample.

Concentration of the Contamination

Large foodstuff sample analysis requires the use of a large volume of diluent (liters of water) to facilitate separation of the contamination from the foodstuff. Some methodologies currently used to achieve this may employ centrifugation or dialysis to concentrate the microbial and chemical targets present. Flash evaporation or solid support affinity columns may be used to concentrate the chemical contaminant. Both methodologies pose problematic for use. Centrifugation is cumbersome and very small microbes (viral) would be difficult to isolate in large volume (liter) solutions. Flash evaporators or solid support affinity columns provide varying concentration efficiencies for each specific chemical. What is needed is a uniform contaminant concentration technique that works for all microbial and chemical contaminants. Furthermore, the concentration technique must be independent of the size of the sample diluent (volume) used.

This invention is a novel method for concentration of all microbial and chemical contaminants by passage of the diluent containing the contaminant, through a cartridge containing sensitized red blood cell (RBC) membrane or stroma. The stroma in one embodiment of the assay is Rh POS (R₂R₂) RBC membranes that were sensitized by the following molecule pair (MP):

IgG anti contaminant—IgG anti-D (Rh)

Mab #1-Mab #2

In the previous blood safety example, the microbial surface molecule used as a target is a peptidoglycan (PG) specific antibody which reacts with both Gram-positive and Gram-negative microorganisms and soluble PG in solution. Any surface target may be utilized.

Mab #1 attached to Mab #2 where Mab #1 possesses specificity for the microbial particle or the chemical molecule. Mab #2 possesses specificity for the D (Rh) site on the Rh POS RBC. This antibody is required to sensitize the RBC (Rh POS) without fixing complement, a phenomenon known to those skilled in the art.

The cartridge volume is directly related to the volume of diluent assayed. For most applications the volume should range form 5 to 50 ml (sufficient volume to hold the proper amount of sensitized packed RBC stroma). The column, filled with stroma, possesses a large porosity membrane or fritted disk on both ends that will retain the sensitized RBC stroma as the large volume diluent solution is passed through the cartridge. To avoid gravity and diluent flow pressure packing of the stroma, the diluent is fed in an antigravity manner (vertical oriented cartridge with inflow of diluent in bottom). The flow rate must be empirically determined, however, a typical rate should range from 1 to 10 ml/minute up to 100 ml/minute (from 60 milliliters to 6 liters flow through per hour). All aspects of cartridge design and operation parameters must assure binding of all contamination targets to the stroma.

The choice of RBC membrane as a capture matrix was not accidental. It is known that antigen/antibody interactions fix immune complement under certain conditions. It is also known that this antigen/antibody complex, where the antigen is affixed to a RBC or RBC membrane, in proximity to the RBC lipid membrane will support the fixation and greatest activation of complement possible.

In a novel manner, Membrane Assisted Complement Mediated Signal Amplification (MACMSA) reverses the situation wherein the antibody with contaminant target specificity is attached to the RBC membrane. Thus, complexation of the microbe and chemical contaminant with the appropriate specificity MP RBC stroma in the presence of immune complement and its required cofactors will allow fixation and maximal activation of the immune complement cascade.

In this invention, the complement is activated via the Classical Complement Pathway requiring both Ca++ and Mg++ as a cofactor. Another pathway present is the Alternate Complement Pathway, which requires Mg++ and activates complement via a different mechanism (based on the presence of carbohydrates in the bacterial cell walls). This pathway possesses a higher Mg++ requirement than the Classical Pathway.

Generation of an Amplified Signal by the Presence of Captured Contamination Targets by Activation of the Classical Complement Pathway

As previously stated, the contamination targets are concentrated by passage of the diluent solution through the stroma cartridge and by attachment of the immunogenic contamination target to the MP on the MP RBC. The target/stroma complex, present in approximately 5 to 25 ml volume of water, is resuspended in buffer and the following is added:

Immune complement (appropriately diluted)

Ca⁺⁺ and Mg⁺⁺ pH ˜7.2

The complement filled target loaded stroma cartridge is incubated at room temperature to permit the fixation and activation of the complement cascade. This results in generation of several different complement cascade activation products at significantly amplified levels. In one MACMSA embodiment, the C4a peptide is theoretically produced at a ratio of 10,000:1 [ratio of C4a:target]. In other assay embodiments, any other complement activation product may be used as a signal; however, none are amplified to the extent of the C4a peptide. Table VII presents some of the possible complement activation products signals. Each activation product via sandwich ELISA is labeled with alkaline phosphatase and sensitive chemiluminescent substrates are used to detect and quantify the activation products present. As depicted in Table VII, detection of the C4a peptide produced theoretically supports sensitive contamination target detection in very large foodstuff samples.

MACMSA Food Safety Assay Characteristics

The basics of the assay have been herein presented. The assay can be fully automated or configured as a semi-automated assay. The total assay time will range from 1.5 to 3.0 hours, dependent on the sample size and volume of diluent used. The assay will detect most microbes and most chemical contaminants preprogrammed into it by using Mab cocktail mixtures with the requirement for a high affinity and avidity Mab with specificity for the target.

Table VIII depicts a comparison between PCR and MACMSA analysis of bacterial contaminated water sources. PCR routinely requires enrichment to function in this application area. Sometimes immunomagnetic separation (IMS) by antibody coated magnetic beads is used to concentrate the bacterial contamination for PCR analysis. Understanding the downsides in Mab coating of magnetic beads, this approach should be less favored. The value of MACMSA diagnostic processes lies in the ability to perform target collection, target concentration, and target signal generation in a single step, namely loading the membrane filled cartridge.

This contaminated water analysis chart closely resembles the analysis of the solid food material diluent previously discussed.

Table IX represents an explanation of the current sensitivity levels set by regulatory agencies for chemical testing. Herein, PPB (parts per billion) represents the lowest level of sensitivity currently obtainable in chemical analysis of a sample. Most chemicals are regulated in ingested foodstuffs to PPB levels only. Examples include: municipal water testing, and Aflatoxin B1 detection in tobacco processates. Note that MACMSA supports unprecedented levels of sensitivity in the detection of chemical contamination.

Discrimination of Live vs. Dead Microorganisms by mRNA TPA Analysis Process

The assay will detect microbes that are alive or dead. Depending on the foodstuff and demands on the assay, it may be necessary to confirm the presence of the live microbial contaminant.

All live microbial cells, bacteria, fungi, etc. possess mRNA, a requirement for live. Dead cells are devoid of mRNA due to their inability to produce it and the lability of the mRNA that was present in the once live cell. Discrimination of the two can be achieved by the use of HP's mRNA RP-TFO TPA assay process, wherein post mRNA isolation by conventional techniques, a non-duplex hairpin (reverse polarity-triplex forming oligonucleotide—RP TFO) is hybridized to the isolated mRNA and a single strand 3′→5′ acting exoribonuclease or other is added to destroy non-specific mRNA, while the triplex formed by the complexation of the target mRNA and the specific RP-TFO is resistant to the exonuclease. mRNA TPA is presented in related patents and will not be discussed further. The protected target complex may be sensitively detected using any number of strategies for signal amplification (see inclusive documents).

The basic assay would involve parallel stromal cartridge concentration of the bacteria, followed by chloroquine treatment to release the captured bacteria in a minimum volume and finally automated mRNA analysis of the collected bacterial contaminants.

TABLE II
CHARACTERIZATION OF ADVANCED BIOLOGICAL WARFARE
DIAGNOSTIC PROCESSES

| Method Characterization | MACMSA | RNA TPA | PCR/Thermal Cyclers |
|---|---|---|---|
| Range of Detectable Agents | Combined microbe/toxin detection | Microbe detection | Microbe detection |
| | and chemical agent detection | | |
| Pathologic Target | Any Immunogenic Moiety | Microbe possessing RNA | Microbe possessing DNA |
| Requirements | (microbe, microbial toxin, or | | (RT PCR of RNA possible) |
| | chemical) | | |
| | Must possess target-specific | | |
| | monoclonal antibody | | |
| Use of DNA Amplification | NO | NO | YES |
| Use of Signal Amplification | YES | YES | NO |
| Characterization of Assay | Direct assay for complement | Direct RNA analysis with use | DNA amplification with |
| and Signal | fixation with use of sensitive | of sensitive chemiluminescent | direct fluorescence read out |
| | chemiluminescent substrate | substrate | of amplified target signal |
| Sample Size | Very large to smaller samples | Very large to smaller samples | Limited to less than 1 mcg. |
| | | | of DNA (lower NG amounts |
| | | | best for assay) |
| Preprocessing Step | Automated sample concentration | Automated sample | Automated DNA extraction |
| Requirements | or, if larger sample required, | concentration or, if larger | (from microbe) module |
| | minimal manual steps required | sample required, minimal | |
| | | manual steps required | |
| Addition of Assay Reagents | Automated addition of | Automated RNA extraction | Automated addition |
| | complement reagent to the target | | of PCR reagent |
| | bound MP RBC stroma | | |
| Assay for: | Automated assay for moiety | Automated direct RNA | Automated |
| | proportionate to extent of | analysis and assay for | Real Time PCR |
| | complement fixation (reflecting | microbial agent RNA by RNA | |
| | extent of target presence) | TPA process | |
| Methods Introduced to | NTE (only target generates signal) | TPA uses hairpin structures | NONE |
| Increase Specificity | (dirt and normal non-specific | (DNA) to protect the target, | |
| (no false positives) | target material will not generate or | followed by enzyme treatment | |
| | inhibit signal) | to destroy all non-specific | |
| | | RNA | |
| Methods Introduced to | Clinically Relevant/ | Clinically Relevant/ | NONE/NO ATTEMPT |
| Increase Sensitivity | Large Environmental or Other | Large Environmental or | to test more than |
| (no false negatives) | Sample Size Preferred | Other Sample Size | a pinch of hay |
| | (Test Entire Haystack) | Preferred | |
| | | (Test Entire Haystack) | |
| | Use of Signal Amplification | RNA Assay takes | |
| | via Fixation & Activation of | advantage of thousands of | |
| | the Complement Cascade | mRNA molecules | |
| | | produced per microbe | |
| | | target | |
| | Use of Sensitive | Use of Sensitive | |
| | Chemiluminescent Substrate | Chemiluminescent | |
| | | Substrate | |
| Lower Sensitivity Limits | Theoretical 10 to 100 Microbial or | Theoretical 1 to 10 Microbial | 40 to 60 Microbial Targets |
| | Chemical Targets in Large | Targets in Large | in Minimal |
| | Sample Volumes | Sample Volumes | Sample Size |
| | (Test Entire Haystack) | (Test Entire Haystack) | (Test a Pinch of Hay) |
| Stage in Infection or | Earliest in Exposure (Haystack | Earliest in Exposure(Haystack | Later in Exposure |
| Exposure Course in which | Processing ™) | Processing ™) | (insufficient sample size |
| Agent is detected | | | tested) |
| Level of Assay Complexity | +1 | +4 | +1 |
| (+1 [LO] +4 [HI]) Overall | | | |
| Process Complexity | +1 | +3 | +1 |
| Capability to Totally | YES | YES | YES |
| Automate | | | |
| Sophistication of Lab | NO | NO | NO |
| Equipment | | | |
| Requirement of Lab | MINOR | MINOR | MINOR |
| Facility | | | |
| Assay Time | 60-90 minutes | 60-90 minutes | 20 minutes |

TABLE III
TYPE AND THEORETICAL SIZE OF SAMPLE
APPROPRIATE FOR ANALYSIS OF A
BIOLOGICAL/CHEMICAL AGENT EXPOSURE
DIAGNOSTIC

| | Maximum* | | Minimum | |
|---|---|---|---|---|
| Sample | Sample Volume | | Sample Volume | |
| Environmental | | | | |
| Water | 10 | liters | 10 | milliliters |
| Soil | 1-2.5 | Kilograms | 1-50 | milligrams |
| Ingestibles | 5-10 | grams | 0.5-1.0 | microgram |
| Air | 500-1000 | cu. ft. | 1-10 | cu. ft. |
| Clinical (all body fluids) | | | | |
| Urine | 0.5-1.0 | liter | 10-50 | microliters |
| Cerebrospinal | 5-10 | milliliters | 10-50 | microliters |
| Plasma | 250-500 | milliliters | 10-50 | microliters |

| | | |
|---|---|---|
| Sputum | Milliliter amounts | Microliter amounts |
| Nasal | Multiple swabs | Single swabs or lavage |
| | or lavage (large | (small volume/ |
| | volume/100 milliliters) | 50 microliters) |

*provides highest sensitivity

TABLE IV
CDC Classification Of Agents Of Biological Warfare

| | | Diagnostic Assay | |
|---|---|---|---|
| Category And Characteristics | Type | RNA TPA | MACMSA |
| CATEGORY A | | | |
| Easily disseminated or | BACTERIUM | | |
| transmitted person to person | Bacillus anthracis (anthrax) | ✓ | ✓ |
| Cause high mortality | Yersinia pestis (plague) | ✓ | ✓ |
| Potential for major public health | Francisella tularensis | ✓ | ✓ |
| impact | (tularaemia) | | |
| May cause public panic and | VIRUS | | |
| social disruption | Variola major (smallpox) | ✓ | ✓ |
| Requires special action for | Filoviruses | | |
| public health preparedness | Ebola (hemorrhagic fever) | ✓ | ✓ |
| Diagnostic technology | Marburg (hemorrhagic | ✓ | ✓ |
| Stockpile vaccines and | fever) | | |
| drugs | Arenaviruses | | |
| Support development of | Lassa (lassa fever) | ✓ | ✓ |
| both of the above | Junin (argentine | ✓ | ✓ |
| | hemorrhagic fever) | | |
| | Related viruses | ✓ | ✓ |
| CATEGORY B | | | |
| Moderately easy to disseminate | BACTERIA | | |
| Cause moderate morbidity | Coxiella burnetti (Q fever) | ✓ | ✓ |
| Cause low mortality | Brucella species (brucellosis) | ✓ | ✓ |
| Requires specific enhancement | Burkholderia mallei | ✓ | ✓ |
| of CDCs | (glanders) | | |
| Diagnostic capacity | VIRUS | | |
| Disease surveillance | Alpha viruses | | |
| | Venezuelan | ✓ | ✓ |
| | encephalomyelitis | | |
| | Eastern equine | ✓ | ✓ |
| | encephalomyelitis | | |
| | Western equine | ✓ | ✓ |
| | encephalomyelitis | | |
| | Toxins | | |
| | Ricin toxin from Castor | | ✓ |
| | Bean | | |
| | Epsilon toxin from C. | | ✓ |
| | perfringens | | |
| | Enterotoxin from | | ✓ |
| | Staphylococcus | | |
| | enterotoxin B | | |
| | BACTERIA (Food or Water Borne) | | |
| | Salmonella species | ✓ | ✓ |
| | Shigella dysenteriae | ✓ | ✓ |
| | E. coli 0157: H7 | ✓ | ✓ |
| | Vibrio cholerae | ✓ | ✓ |
| | Cryptosporidium parvum | ✓ | ✓ |
| CATEGORY C | | | |
| Availability | BACTERIA | | |
| Ease of production and | Multi drug resistant | ✓ | ✓ |
| dissemination | tuberculosis | | |
| Potential for high morbidity | VIRUS | | |
| Potential for high mortality | Nipah | ✓ | ✓ |
| Major health impact | Hanta | ✓ | ✓ |
| Requires research in | Tickborne hemorrhagic fever | ✓ | ✓ |
| Disease detection | Tickborne encephalitis | ✓ | ✓ |
| Diagnosis | Yellow fever | ✓ | ✓ |
| Treatment | | | |
| Prevention | | | |

Table V: BCWA Complement Fixation Diagnostic Assays and their Sensitivities

TABLE V
BCWA COMPLEMENT FIXATION DIAGNOSTIC
ASSAYS AND THEIR SENSITIVITIES

| | | | | Theoretical Assay¹ | |
|---|---|---|---|---|---|
| | | Signal | | Sensitivity (Target | |
| BCWA | | Amplification | | Numbers²) Using: | |
| Target | Assay | Per Target | | C3a Assay | C4a Assay |
| Bacteria | MACMSA | C3a | >100,000³ | 1-100 | — |
| | | C4a | — | | |
| Bacterial | MACMSA | C3a | 500 | 2-100 | 1-100 |
| Toxins | | C4a | 10,000 | | |
| Virus | MACMSA | C3a | 10,000 | 1-100 | 1-100 |
| | | C4a | 10,000 | | |
| Immunogenic | MACMSA | C3a | 500 | 2-200 | 1-100 |
| Chemicals | | C4a | 10,000 | | |

| | | | |
|---|---|---|---|
| Bacteria | mRNA-TPA | 1,000⁴ | 1-100 |
| Virus | mRNA-TPA | 1,000⁵ | 1-100 |

¹Based on Alkaline Phosphatase Assay of either C3a or C4a peptide or mRNA molecule assay, wherein 1,000 AP molecules and the 1,2 dioxetane chemiluminescent substrates provide detection.
²Range result to account for variable assay.
³Increased due to activation of Alternate Pathway of complement fixation (C3a only, no C4a).
⁴Microbe mRNA molecules per infected cell.
⁵Need molecular signal amplification technology.

TABLE VI
ALGORITHM FOR AFB1 TESTING IN
TOBACCO PROCESSING

| | | | | QC |
|---|---|---|---|---|
| Test | | | Each Process | Volatilization |
| Material | Raw Tobacco | | Step | Testing |
| Analyte | Aspergillus | Soluble | Soluble | Soluble |
| | Sp. Assay | AFB1 | AFB1 | AFB1 |
| Diagnostic | CMSA | MACMSA | MACMSA | MACMSA |
| Process | Alternate | Classical | Classical | Classical |
| | Complement | Complement | Complement | Complement |
| | Fixation | Fixation | Fixation | Fixation |
| | Pathway | Pathway | Pathway | Pathway |
| Theoretic | Few Micro- | Few | Few | Few |
| Sensitivity | organisms | Molecules | Molecules | Molecules |
| Levels | (10 or | (100 or | (100 or | (100 or |
| | more) | more) | more) | more) |
| Volume of | No | No | No | No |
| Batch | Limitation | Limitation | Limitation | Limitation |
| Aliquot | | | | |
| Tested | | | | |
| Non-specific | None | None | None | None |
| Signal* | | | | |
| Background | | | | |

*none generated by non-specific analyte

TABLE VII
Internet Web Source: http://www.epa.gov/safewater/mcl.html
EPA United States Environmental Protection Agency NATIONAL PRIMARY DRINKING WATER STANDARDS

| | | | | |
|---|---|---|---|---|
| | MCLG¹ | MCL or TT¹ | Potential Health Effects from Ingestion of | Sources of contaminant in drinking |
| Contaminant | (mg/L)² | (mg/L)² | Water | water |

MICROORGANISMS

| | | | | |
|---|---|---|---|---|
| Cryptosporidium | as of | as of | Gastrointestinal illness (e.g., diarrhea, vomiting, | Human and animal fecal waste |
| | Jan. 01, 2002: | Jan. 01, 2002: | cramps) | |
| | zero | TT³ | | |
| Giardia lamblia | zero | TT³ | Gastrointestinal illness (e.g., diarrhea, vomiting, | Human and animal fecal waste |
| | | | cramps) | |
| Heterotrophic plate count | n/a | TT³ | HPC has no health effects, but can indicate how | HPC measures a range of bacteria |
| (HPC) | | | effective treatment is at controlling | that are naturally present in the |
| | | | microorganisms. | environment |
| Legionella | zero | TT³ | Legionnaire's Disease, commonly known as | Found naturally in water; multiplies |
| | | | pneumonia | in heating systems |
| Total Coliforms | zero | 5.0%⁴ | Used as an indicator that other potentially | Coliforms are naturally present in the |
| (including fecal | | | harmful bacteria may be present⁵ | environment; fecal coliforms and E. |
| coliform and | | | | coli come from human and animal |
| E. Coli) | | | | fecal waste. |
| Turbidity | n/a | TT³ | Turbidity is a measure of the cloudiness of | Soil runoff |
| | | | water. It is used to indicate water quality and | |
| | | | filtration effectiveness (e.g., whether disease- | |
| | | | causing organisms are present). Higher turbidity | |
| | | | levels are often associated with higher levels of | |
| | | | disease-causing microorganisms such as | |
| | | | viruses, parasites and some bacteria. These | |
| | | | organisms can cause symptoms such as nausea, | |
| | | | cramps, diarrhea, and associated headaches. | |
| Viruses (enteric) | zero | TT³ | Gastrointestinal illness (e.g., diarrhea, vomiting, | Human and animal fecal waste |
| | | | cramps) | |

DISINFECTANTS AND DISINFECTION BYPRODUCTS

| | | | | |
|---|---|---|---|---|
| Bromate | as of | as of | Increased risk of cancer | Byproduct of drinking water |
| | Jan. 01, 2002: | Jan. 01, 2002: | | disinfection |
| | zero | 0.010 | | |
| Chloramines (as Cl₂) | as of | as of | Eye/nose irritation; stomach discomfort, anemia | Water additive used to control |
| | Jan. 01, 2002: | Jan. 01, 2002: | | microbes |
| | MRDL = 4¹ | MRDL = 4.0¹ | | |
| Chlorine (as Cl₂) | as of | as of | Eye/nose irritation; stomach discomfort | Water additive used to control |
| | Jan. 01, 2002: | Jan. 01, 2002: | | microbes |
| | MRDLG = 4¹ | MRDL = 4.0¹ | | |
| Chlorine dioxide | as of | as of | Anemia; | Water additive used to control |
| (as ClO₂) | Jan. 01, 2002: | Jan. 01, 2002: | infants & young children: nervous system | microbes |
| | MRDLG = 0.8¹ | MRDL = 0.8¹ | effects | |
| Chlorite | as of | as of | Anemia; | Byproduct of drinking water |
| | Jan. 01, 2002: | Jan. 01, 2002: | infants & young children: nervous system | disinfection |
| | 0.8 | 1.0 | effects | |
| Haloacetic acids (HAA5) | as of | as of | Increased risk of cancer | Byproduct of drinking water |
| | Jan. 01, 2002: | Jan. 01, 2002: | | disinfection |
| | n/a⁶ | 0.060 | | |
| | none⁷ | 0.10 | | |
| Total Trihalomethanes | as of | as of | Liver, kidney or central nervous system | Byproduct of drinking water |
| (TTHMs) | Jan. 01, 2002: | Jan. 01, 2002: | problems; increased risk of cancer | disinfection |
| | n/a⁶ | 0.080 | | |

INORGANIC CHEMICALS

| | | | | |
|---|---|---|---|---|
| Antimony | 0.006 | 0.006 | Increase in blood cholesterol; decrease in blood | Discharge from petroleum refineries; |
| | | | glucose | fire retardants; ceramics; electronics; |
| | | | | solder |
| Arsenic | none⁷ | 0.05 | Skin damage; circulatory system problems; | Erosion of natural deposits; runoff |
| | | | increased risk of cancer | from glass & electronics production |
| | | | | wastes |
| Asbestos | 7 million | 7 MFL | Increased risk of developing benign intestinal | Decay of asbestos cement in water |
| (fiber < 10 micrometers) | fibers per liter | | polyps | Mains; erosion of natural deposits |
| Barium | 2 | 2 | Increase in blood pressure | Discharge of drilling wastes; |
| | | | | discharge from metal refineries; |
| | | | | erosion of natural deposits |
| Beryllium | 0.004 | 0.004 | Intestinal lesions | Discharge from metal refineries and |
| | | | | coal-burning factories; discharge |
| | | | | from electrical, aerospace, and |
| | | | | defense industries |
| Cadmium | 0.005 | 0.005 | Kidney damage | Corrosion of galvanized pipes; |
| | | | | erosion of natural deposits; discharge |
| | | | | from metal refineries; runoff from |
| | | | | waste batteries and paints |
| Chromium (total) | 0.1 | 0.1 | Some people who use water containing | Discharge from steel and pulp mills; |
| | | | chromium well in excess of the MCL over | erosion of natural deposits |
| | | | many years could experience allergic dermatitis | |
| Copper | 1.3 | TT⁸; | Short term exposure: Gastrointestinal distress. | Corrosion of household plumbing |
| | | Action | Long term exposure: Liver or kidney damage. | systems; erosion of natural deposits |
| | | Lever = 1.3 | People with Wilson's Disease should consult | |
| | | | their personal doctor if their water systems | |
| | | | exceed the copper action level. | |
| Cyanide (as free cyanide) | 0.2 | 0.2 | Nerve damage or thyroid problems | Discharge from steel/metal factories; |
| | | | | discharge from plastic and fertilizer |
| | | | | factories |
| Fluoride | 4.0 | 4.0 | Bone disease (pain and tenderness of the | Water additive which promotes |
| | | | bones); Children may get mottled teeth | stron teeth; erosion of natural |
| | | | | deposits; discharge from fertilizer |
| | | | | and aluminum factories |
| Lead | zero | TT⁸; Action | Infants and children: Delays in physical or | Corrosion of household plumbing |
| | | Level = 0.015 | mental development. Adults: Kidney problems; | systems; erosion of natural deposits |
| | | | high blood pressure | |
| Mercury (inorganic) | 0.002 | 0.002 | Kidney damage | Erosion of natural deposits; discharge |
| | | | | from refineries and factories; runoff |
| | | | | from landfills and cropland |
| Nitrate (measured as | 10 | 10 | “Blue baby syndrome” in infants under six | Runoff from fertilizer use; leaching |
| Nitrogen) | | | months - life threatening without immediate | from septic tanks, sewage; erosion of |
| | | | medical attention. | natural deposits |
| | | | Symptoms: Infant looks blue and has shortness | |
| | | | breath. | |
| Selenium | 0.05 | 0.05 | Hair or fingernail loss; numbness in fingers or | Discharge from petroleum refineries; |
| | | | toes; circulatory problems | erosion of natural deposits; discharge |
| | | | | from mines |
| Thallium | 0.0005 | 0.002 | Hair loss; changes in blood; kidney, intestine, or | Leaching from ore-processing sites; |
| | | | liver problems | discharge from electronics, glass, and |
| | | | | pharmaceutical companies |

ORGANIC CHEMICALS

| | | | | |
|---|---|---|---|---|
| Acrylamide | zero | TT⁹ | Nervous system or blood problems; increased | Added to water during |
| | | | risk of cancer | sewage/waste water treatment |
| Alachlor | zero | 0.002 | Eye, liver, kidney or spleen problems, anemia; | Runoff from herbicide used on row |
| | | | increased risk of cancer | crops |
| Atrazine | 0.003 | 0.003 | Cardiovascular system problems; reproductive | Runoff from herbicide used on row |
| | | | difficulties | crops |
| Benzene | zero | 0.005 | Anemia; decrease in blood platelets; increased | Discharge from factories; leaching |
| | | | risk of cancer | from gas storage tanks and landfills |
| Benzo(a)pyrene (PAHs) | zero | 0.0002 | Reproductive difficulties; increased risk of | Leaching from linings of water |
| | | | cancer | storage tanks and distribution lines |
| Carbofuran | 0.04 | 0.04 | Problems with blood or nervous system; | Leaching of soil fumigant used on |
| | | | reproductive difficulties | rice and alfalfa |
| Carbon | zero | 0.005 | Liver problems; increased risk of cancer | Discharge from chemical plants and |
| tetrachloride | | | | other industrial activities |
| Chlordane | zero | 0.002 | Liver or nervous system problems; increased | Residue of banned termiticide |
| | | | risk of cancer | |
| Chlorobenzene | 0.1 | 0.1 | Liver or kidney problems | Discharge from chemical and |
| | | | | agricultural chemical factories |
| 2,4-D | 0.07 | 0.07 | Kidney, liver, or adrenal gland problems | Runoff from herbicide used on row |
| | | | | crops |
| Dalapon | 0.2 | 0.2 | Minor kidney changes | Runoff from herbicide used on rights |
| | | | | of way |
| 1,2-Dibromo-3- | zero | 0.0002 | Reproductive difficulties; increased risk of | Runoff/leaching from soil fumigant |
| chloropropane (DBCP) | | | cancer | used on soybeans, cotton, pineapples, |
| | | | | and orchards |
| o-Dichlorobenzene | 0.6 | 0.6 | Liver, kidney, or circulatory system problems | Discharge from industrial chemical |
| | | | | factories |
| p-Dichlorobenzene | 0.075 | 0.075 | Anemia; liver, kidney or spleen damage; | Discharge from industrial chemical |
| | | | changes in blood | factories |
| 1,2-Dichloroethane | zero | 0.005 | Increased risk of cancer | Discharge from industrial chemical |
| | | | | factories |
| 1,1-Dichloroethylene | 0.007 | 0.007 | Liver problems | Discharge from industrial chemical |
| | | | | factories |
| cis-1,2-Dichloroethylene | 0.07 | 0.07 | Liver problems | Discharge from industrial chemical |
| | | | | factories |
| trans-1,2-Dichloro- | 0.1 | 0.1 | Liver problems | Discharge from industrial chemical |
| ethylene | | | | factories |
| Dichloromethane | zero | 0.005 | Liver problems; increased risk of cancer | Discharge from pharmaceutical and |
| | | | | chemical factories |
| 1,2-Dichloropropane | zero | 0.005 | Increased risk of cancer | Discharge from industrial chemical |
| | | | | factories |
| Di(2-ethylhexyl) adipate | 0.4 | 0.4 | General toxic effects or reproductive difficulties | Leaching from PVC plumbing |
| | | | | systems; discharge from chemical |
| | | | | factories |
| Di(2-ethylhexyl) | zero | 0.006 | Reproductive difficulties; liver problems; | Discharge from rubber and chemical |
| phthalate | | | increased risk of cancer | factories |
| Dinoseb | 0.007 | 0.007 | Reproductive difficulties | Runoff from herbicide used on |
| | | | | soybeans and vegetables |
| Dioxin (2,3,7,8-TCDD) | zero | 0.00000003 | Reproductive difficulties; increased risk of | Emissions from waste incineration |
| | | | cancer | and other combustion; discharge |
| | | | | from chemical factories |
| Diquat | 0.02 | 0.02 | Cataracts | Runoff from herbicide use |
| Endothall | 0.1 | 0.1 | Stomach and intestinal problems | Runoff from herbicide use |
| Endrin | 0.002 | 0.002 | Nervous system effects | Residue of banned insecticide |
| Epichlorohydrin | zero | TT⁹ | Stomach problems; reproductive difficulties; | Discharge from industrial chemical |
| | | | increased risk of cancer | factories; added to water during |
| | | | | treatment process |
| Ethylbenzene | 0.7 | 0.7 | Liver or kidney problems | Discharge from petroleum refineries |
| Ethelyne dibromide | zero | 0.00005 | Stomach problems; reproductive difficulties; | Discharge from petroleum refineries |
| | | | increased risk of cancer | |
| Glyphosate | 0.7 | 0.7 | Kidney problems; reproductive difficulties | Runoff from herbicide use |
| Heptachor | zero | 0.0004 | Liver damage; increased risk of cancer | Residue of banned termiticide |
| Heptachlor epoxide | zero | 0.0002 | Liver damage; increased risk of cancer | Breakdown of hepatachlor |
| Hexachlorobenzene | zero | 0.001 | Liver or kidney problems; reproductive | Discharge from metal refineries and |
| | | | difficulties; increased risk of cancer | agricultural chemical factories |
| Hexachlorocyclo- | 0.05 | 0.05 | Kidney or stomach problems | Discharge from chemical factories |
| pentadiene | | | | |
| Lindane | 0.0002 | 0.0002 | Liver or kidney problems | Runoff/leaching from insecticide |
| | | | | used on cattle, lumber, gardens |
| Methoxychlor | 0.04 | 0.04 | Reproductive difficulties | Runoff/leaching from insecticide |
| | | | | used on fruits, vegetables, alfalfa, |
| | | | | livestock |
| Oxamyl (Vydate) | 0.2 | 0.2 | Slight nervous system effects | Runoff/leaching from insecticide |
| | | | | used on apples, potatoes, and |
| | | | | tomatoes |
| Polychlorinated | zero | 0.0005 | Skin changes; thymus gland problems; immune | Runoff from landfils; discharge of |
| biphenyls (PCBs) | | | deficiencies; reproductive or nervous system | waste chemicals |
| | | | difficulties; increased risk of cancer | |
| Pentachlorophenol | zero | 0.001 | Liver or kidney problems; increased risk of | Discharge from wood preserving |
| | | | cancer | factories |
| Picloram | 0.5 | 0.5 | Liver problems | Herbicide runoff |
| Simazine | 0.004 | 0.004 | Problems with blood | Herbicide runoff |
| Styrene | 0.1 | 0.1 | Liver, kidney, and circulatory problems | Discharge from rubber and plastic |
| | | | | factories; leaching from landfills |
| Tetrachloroethylene | zero | 0.005 | Liver problems; increased risk of cancer | Discharge from factories and dry |
| | | | | cleaners |
| Toluene | 1 | 1 | Nervous system, kidney, or liver problems | Discharge from petroleum factories |
| Toxaphene | zero | 0.003 | Kidney, liver, or thyroid problems; increased | Runoff/leaching from insecticide |
| | | | risk of cancer | used on cotton and cattle |
| 2,4,5-TP (Silvex) | 0.05 | 0.05 | Liver problems | Residue of banned herbicide |
| 1,2,4-Trichlorobenzene | 0.07 | 0.07 | Changes in adrenal glands | Discharge from textile finishing |
| | | | | factories |
| 1,1,1-Trichloroethane | 0.20 | 0.2 | Liver, nervous system, or circulatory problems | Discharge from metal degreasing |
| | | | | sites and other factories |
| 1,1,2-Trichloroethane | 0.003 | 0.005 | Liver, kidney, or immune system problems | Discharge from industrial chemical |
| | | | | factories |
| Trichloroethylene | zero | 0.005 | Liver problems; increased risk of cancer | Discharge from petroleum refineries |
| Vinyl chloride | zero | 0.002 | Increased risk of cancer | Leaching from PVC pipes; discharge |
| | | | | from plastice factories |
| Xylenes (total) | 10 | 10 | Nervous system damage | Discharge from petroleum factories; |
| | | | | discharge from chemical factories |

RADIONUCLIDES

| | | | | |
|---|---|---|---|---|
| Alpha particles | none7 | 15 picocuries | Increased risk of cancer | Erosion of natural deposits |
| | | per Liter | | |
| | | (pCi/L) | | |
| Beta particles and photon | none7 | 4 millirems | Increased risk of cancer | Decay of natural and man-made |
| emitters | | per year | | deposits |
| Radium 226 and Radium | none7 | 5 pCi/L | Increased risk of cancer | Erosion of natural deposits |
| Uranium | as of | as of | Increased risk of cancer; kidney toxicity | Erosion of natural deposits |
| | Dec. 8, 2003: | Dec. 8, 2003: | | |
| | zero | 30 ug/L | | |

NOTES
¹Definitions:
Maximum Contaminant Level (MCL)—The highest level of a contaminant that is allowed in drinking water. MCLs are set as close to MCLGs as feasible using the best available treatment technology and taking cost into consideration. MCLs are enforceable standards.
Maximum Contaminant Level Goal (MCLG)—The level of a contaminant in drinking water below which there is no known or expected risk to health. MCLGs allow for a margin of safety and are non-enforceable public health goals.
Maximum Residual Disinfectant Level (MRDL)—The highes level of a disinfectant allowed in drinking water. There is convincing evidence that addition of a disinfectant is necessary for control of microbial contaminants.
Maximum Residual Disinfectant Level Goal (MRDLG)—The level of a drinking water disinfectanct below which there is no known or exprected risk to health. MRDLGs do not reflect the benefits of the use of disinfectants to control microbial contaminants.
Treatment Technique—A required process intended to reduce the level of a contaminant in drinking water.
²Units are in milligrams per liter (mg/L) unless otherwise noted. Milligrams per liter are equivalent to parts per million.
³EPA's surface water treatment rules require systems using surface water or ground water under the direct influence of surface water to (1) disinfect their water, and (2) filter their water or meet criteria for avoiding filtration so that the following contaminants are controlled at the following levels:
Cryptosporidum: (as of Jan. 1, 2002) 99% removal/inactivation
Giardia lamblia: 99.9% removal/inactivation
Viruses: 99.99% removal/inactivation
Legionella: No limit, but EPA believes that if Giardia and viruses are removed/inactivated, Legionella will also be controlled.
Turbidity: At no time can turbidity (cloudiness of water) go above 5 nephelolometric turbidity units (NTU); systems that filter must ensure that the turbidity go no higher than 1 NTU (0.5 NTU for conventional or direct filtration) in at least 95% of the daily samples in any month. As of Jan. 1, 2002, turbidity may never exceed 1 NTU, and must not exceed 0.3 NTU in 95% of daily samples in any month.
HPC: No more than 500 bacterial colonies per milliliter.
⁴No more than 5.0% samples total coliform-positive in a month. (For water systems that collect fewer than 40 routine samples per month, no more than one sample can be total coliform-positive). Every sample that has total coliforms must be analyzed for fecal coliforms. There may not be any fecal coliforms or E. coli.
⁵Fecal coliform and E. coli are bacteria whose presence indicates that the water may be contaminated with human or animal wastes. Disease-causing microbes (pathogens) in these wastes can cause diarrhea, cramps, nausea, headaches, or other symptoms. These pathogens may pose a special health risk for infants, young children, and people with severely compromised immune systems.
⁶Although there is no collective MCLG for this contaminant group, there are individual MCLGs for some of the individual contaminants:
Haloacetic acids: dichloracetic acid (zero); trichloroacetic acid (0.3 mg/L). Monochloroacetic acid, bromoacetic acid, and dibromoacetic acid are regulated with this group but have no MCLGs.
Trihalomethanes: bromodichloromethane (zero); bromofrom (zero); dibromochloromethane (0.06 mg/L). Chloroform is regulated with this group but has no MCLG.
⁷MCLGs were not established before the 1986 Amendments to the Safe Drinking Water Act. Therefore, there is no MCLG for this contaminant.
⁸Lead and copper are regulated by a Treatment Technique that requires systems to control the corrosiveness of their water. If more than 10% of tap water samples exceed the action level, water systems must take additional steps. For copper, the action level is 1.3 mg/L, and for lead is 0.015 mg/L.
⁹Each water system must certify, in writing, to the state (using third-party or manufacturer's certification) that when acrylamide and epichlorohydrin are used in drinking water systems, the combination (or product) of dose and monomer level does not exceed the levels specified, as follows:
Acrylamide = 0.05% dosed at 1 mg/L (or equivalent)
Epichlorohydrin = 0.01% dosed at 20 mg/L (or equivalent)

TABLE VIII.1
Internet Web Source: http://www.cabq.gov/progress/EP02WATQ.html
Albuquerque 2000 Progress Report
ENVIRONMENTAL PROTECTION & ENHANCEMENT
Desired Community Condition
Air, land and water systems protect health and safety.
Indicator
Water Quality
Unregulated Substances Tested For and Not Detected

| | | | |
|---|---|---|---|
| Aldicarb | Chloral Hydrate | 1,1-Dichloropropene | Naphthalene |
| Aldicarb sulfone | Chloroethane | 1,3-Dichloropropene | Propachlor |
| Aldicarb sulfoxide | Chloromethane | Dieldrin | n-Propylbenzene |
| Aldrin | o-Chlorotoluene | Fluorotrichloromethane | Sulfate |
| Bromobenzene | p-Chlorotoluene | Hexachlorobutadiene | 1,1,1,2- |
| | | | Tetrachloroethane |
| Bromochloromethane | Dibromomethane | 3-Hydroxycarbofuran | 1,1,2,2- |
| | | | Tetrachloroethane |
| Bromomethane | Dicamba | Isopropylbenzene | 1,2,3-Trichlorobenzene |
| (methyl bromide) | | | |
| Butachlor | Dichlorodifluoromethane | p-Isopropyltoluene | 1,2,3-Trichloropropane |
| sec-Butylbenzene | 1,1-Dichloroethane | Methomyl | 1,2,4-Trimethylbenzene |
| n-Butylbenzene | 2,2-Dichloropropane | Metolachlor | 1,3,5-Trimethylbenzene |
| tert-Butylbenzene | 1,3-Dichloropropane | Metribuzin | Total Organic Halides |
| Carbaryl | | | |

TABLE IX
Internet Web Source: http://www.stimson.org/cwc/bwagent.htm
Chemical and Biological Weapons Nonproliferation Project
Biological Weapons Agents
Table 1: Characteristics and Symptoms of Some Anti-Human Biological Agents¹

| Agent | | Rate of | Effective | | |
|---|---|---|---|---|---|
| Type | Name of Agent | Action | Dosage | Symptoms/Effects | Prophylaxis/Treatment |
| Bacteria | Bacillus | Incubation: | 8,000 to | Fever and fatigue; | Treatable, if antibiotics |
| | anthracis | 1 to 6 days | 50,000 | often followed by a | administered prior to |
| | Causes anthrax | Length of | spores | slight improvement, | onset of symptoms |
| | | illness: | | then abrupt onset of | Vaccine available |
| | | 1 to 2 days | | severe respiratory | |
| | | Extremely | | problems; shock; | |
| | | high mortality | | pneumonia and death | |
| | | rate | | within 2 to 3 days | |
| | Yersinia pestis | Incubation: | 100 to 500 | Malaise, high fever, | Treatable, if antibiotics |
| | Causes plague | 2 to 10 days | organisms | tender lymph nodes, | administered within 24 |
| | | Length of | | skin lesions, possible | hours of onset of |
| | | illness: | | hemorrhages, | symptoms |
| | | 1 to 2 days | | circulatory failure, and | Vaccine available |
| | | Variable | | eventual death | |
| | | mortality rate | | | |
| | Brucella suis | Incubation: | 100 to | Flu-like symptoms, | Treatable with antibiotics |
| | Causes | 5 to 60 days | 1,000 | including fever and | No vaccine available |
| | brucellosis | 2% mortality | organisms | chills, headache, | |
| | | rate | | appetite loss, mental | |
| | | | | depression, extreme | |
| | | | | fatigue, aching joints, | |
| | | | | sweating, and possibly | |
| | | | | gastrointestinal | |
| | | | | symptoms. | |
| | Pasturella | Incubation: | 10 to 50 | Fever, headache, | Treatable, if antibiotics |
| | tularensis | 1 to 10 days | organisms | malaise, general | administered early |
| | Causes tularemia | Length of | | discomfort, irritating | Vaccine available |
| | Also known as | illness: | | cough, weight loss | |
| | rabbit fever and | 1 to 3 weeks | | | |
| | deer fly fever | 30% mortality | | | |
| | | rate | | | |
| Rickettsiae | Coxiella burnetti | Incubation: | 10 | Cough, aches, fever, | Treatable with antibiotics |
| | Causes Q-fever | 2 to 14 days | organisms | chest pain, pneumonia | Vaccine available |
| | | Length of | | | |
| | | illness: | | | |
| | | 2 to 14 days | | | |
| | | 1% mortality | | | |
| | | rate | | | |
| Viruses | Variola virus | Incubation: | 10 to 100 | Malaise, fever, | Treatable if vaccine |
| | Causes smallpox | average 12 | organisms | vomiting, headache | administered early |
| | | days | | appear first, followed 2 | Limited amounts of |
| | | Length of | | to 3 days later by | vaccine available |
| | | illness: | | lesions | Note: World Health |
| | | several weeks | | Highly infectious | Organization conducted a |
| | | 35% mortality | | | vaccination campaign |
| | | rate in un- | | | from 1967 to 1977 to |
| | | vaccinated | | | eradicate smallpox. |
| | | individuals | | | |
| | Venezuelan | Incubation: | 10 to 100 | Sudden onset of fever, | No specific therapy exists |
| | equine | 1 to 5 days | organisms | severe headache, and | Vaccine available |
| | encephalitis virus | Length of | | muscle pain | |
| | | illness: | | Nausea, vomiting, | |
| | | 1 to 2 weeks | | cough, sore throat and | |
| | | Low mortality | | diarrhea can follow | |
| | | rate | | | |
| | Yellow fever | Incubation: | 1 to 10 | Severe fever, | No specific therapy exists |
| | virus | 3 to 6 days | organisms | headache, cough, | Vaccine available |
| | | Length of | | nausea, vomiting, | |
| | | illness: | | vascular complications | |
| | | 1 to 2 weeks | | (including easy | |
| | | 5% mortality | | bleeding, low blood | |
| | | rate | | pressure) | |
| Toxins | Botulinum toxin | Time to effect: | .001 | Weakness, dizziness, | Treatable with antitoxin, if |
| | Causes botulism | 34 to 36 hours | microgram | dry throat and mouth, | administered early |
| | Produced by | Length of | per | blurred vision, | Vaccine available |
| | Clostridium | illness: | kilogram of | progressive weakness | |
| | botulinum | 24 to 72 hours | body weight | of muscles | |
| | bacterium | 65% mortality | | Interruption of | |
| | | rate | | neurotransmission | |
| | | | | leading to paralysis | |
| | | | | Abrupt respiratory | |
| | | | | failure may result in | |
| | | | | death | |
| | Saxitoxin | Time to effect: | 10 | Dizziness, paralysis of | |
| | Produced by | minutes to | micrograms | respiratory system, and | |
| | blue-green algae | hours | per | death within minutes | |
| | commonly | Length of | kilogram of | | |
| | ingested by | illness: | body weight | | |
| | shellfish, mussels | Fatal after | | | |
| | in particular | inhalation of | | | |
| | | lethal dose | | | |
| | Ricin | Time to effect: | 3 to 5 | Rapid onset of | No antitoxin or vaccine |
| | Derived from | few hours | micrograms | weakness, fever, | available |
| | castor beans | Length of | per | cough, fluid build-up | |
| | | illness: | kilogram of | in lungs, respiratory | |
| | | 3 days | body weight | distress | |
| | | High mortality | | | |
| | | rate | | | |
| | Staphylococcal | Time to effect: | 30 | Fever, chills, | No specific therapy or |
| | enterotoxin B | 3 to 12 hours | nanograms | headache, nausea, | vaccine available |
| | (SEB) | Length of | per person | cough, diarrhea, and | |
| | Produced by | illness: | | vomiting | |
| | Staphylococcus | Up to 4 weeks | | | |
| | aureus | | | | |

Anti-Plant Biological Agents¹

Rice Blast
Fungal disease causing lesions on leaves
Up to 60% crop losses possible
Stem Rust
Fungal disease affecting cereal crops (e.g., wheat, barley)
Produces pustules on stems, leaves
Can cause significant crop losses
Sugarbeet Curly Top Virus
Viral disease causing dwarfed leaves and swollen veins
Transmitted by beet leafhopper, an insect that can migrate over long distances and attack many different types of plants
Can be controlled through insecticides
Tobacco Mosaic Virus
Viral disease affecting wide range of plant species
Causes leaf blotching in mosaic patterns and stunted growth in younger plants

Anti-Animal Biological Agents¹

Aspergillus
Fungal disease caused by Aspergillus fumigatus infecting poultry
Causes lethargy, loss of appetite, and, in extreme cases, paralysis
Foot and Mouth Disease
Highly contagious viral disease infecting cloven hooved animals (e.g., cattle, pigs, sheep, goats)
Up to 50% mortality rates in young animals; can cause dramatic production decreases in adults
Incubation period generally between 2 and 8 days
Causes fever, loss of appetite, interruption in milk production, blisters (particularly around feet and mouth)
Considered one of the most feared animal diseases because of its high degree of contagiousness and the large number of species affected
Heartwater
Caused by rickettsia Cowdria ruminantium
Disease attacks ruminants, including cattle, sheep, goats and deer
Transmitted by ticks
Mortality rates range from 40% to 100%
Results in loss of appetite, respiratory distress
No effective treatment or vaccine available
Newcastle Disease
Highly contagious viral disease infecting poultry
Causes gastrointestinal, respiratory and nervous problems
Up to 100% mortality rate
Incubation period generally between 5 and 6 days; in severe cases, birds can die within 1 or 2 days
Vaccine available
Rinderpest
Highly contagious viral disease infecting cattle
Also referred to as cattle plague
Spread primarily through direct contact and infected drinking water
Causes fever, frothy saliva, diarrhea
Vaccine available
Sources: U.S. Army Medical Research Institute of Infectious Diseases, Handbook on the Medical Aspects of NBC Defensive Operations, FM February 8-9, 1996; Robert E. Boyle, Biological Warfare: A Historical Perspective, Sandia National Laboratories, February 1998; U.S. Army Medical Research Institute of Infectious Diseases, Medical Management of Biological Casualties, Third Edition, July 1998; Col. David R. Franz et al., “Clinical Recognition and Management of Patients Exposed to Biological Warfare
# in the 21st Century: Biotechnology and the Proliferation of Biological Weapons, (Brassey's, U.K.: London, 1994); Institute for Animal Health, Reports and Publications - 1997, accessed electronically at [http://www.iah.bbsrc.ac.uk/reports/1997/]; United Nations Food and Agriculture Organization, Global Rinderpest Eradication Program, accessed eelctronically at [http://www.fao.org/waicent/faoinfo/agricult/aga/agah/empres/grep.htm].

TABLE X
MACMSA SIGNAL (ACTIVATED CLASSICAL COMPLEMENT
PATHWAY) USED VS. TARGET NUMBERS DETECTED USING
ALKALINE PHOSPHATASE AND SENSITIVE CHEMILUMINESCENT
SUBSTRATES FOR SIGNAL QUANTIFICATION

| | | | | | Minimal | **Minimal |
|---|---|---|---|---|---|---|
| | | | Chemiluminescent | | Target | Target |
| | Number | | Substrate | Total | Detection | Detection |
| Complement | Produced | | Activated To | Light | at 100% | at 1.0% |
| Signal | Per | Assay Used | Produce | Generated | Assay | Assay |
| Component | Target | To Detect Signal | Light | (Units)* | Efficiency | Efficiency |
| C3a | 500 | AP labeling of C3a/ | 3 logs/C3a | 500,000 | 2 | 200 |
| | | chemiluminescent | | | | |
| | | substrate | | | | |
| C1q | 1 | AP labeling of C1q/ | 3 logs/C1q | 1,000 | 1,000 | 100,000 |
| | | chemiluminescent | | | | |
| | | substrate | | | | |
| \overline{C1qrs} | 1 | Use of | 3 logs/\overline{C1qrs} | 1,000 | 1,000 | 100,000 |
| | | chemiluminescent | | | | |
| | | esterase substrate | | | | |
| C4a | 10,000 | AP labeling of C4a/ | 3 logs/C4a | 10,000,000 | 1 | 100 |
| | | chemiluminescent | | | | |
| | | substrate | | | | |
| C5a | >200 | AP labeling of | 3 logs/C5a | 200,000 | 5 | 500 |
| | | C5a/chemiluminescent | | | | |
| | | substrate | | | | |
| MAC | >200 | AP labeling of MAC/ | 3 logs/C5a | 200,000 | 5 | 500 |
| | | chemiluminescent | | | | |
| | | substrate | | | | |

*1,000,000 light units to detect one target using chemiluminescence (published)
**All diagnostic assays possess less than 100% efficiency, herein, 99% assumed assay inefficiency limits will still detect target in the samples.

TABLE XI
WATER TESTING FOR BACTERIAL CONTAMINATION*
PCR VS. MACMSA

| H₂O Test | Number | (PCR) | |
|---|---|---|---|
| Volume | Bacterial | Polymerase | |
| (ml) | Contaminants | Chain Reaction | MACMSA |
| 1 | 1 | Insufficient | Non-detectable |
| | | contamination | |
| | | for enrichment | |
| | | in culture media | |
| 10 | 10 | Insufficient | Perform MACMSA |
| | | contamination for | (Load cartridge flow rate |
| | | enrichment in | 1 ml/minute) |
| | | culture media | C3a Assay |
| | | | Total Time 2.0 |
| | | | hours Assumed |
| | | | 10% efficiency |
| | | | Positive Reaction |
| 100 | 100 | Enrichment in | Perform MACMSA |
| | | culture media | (Load cartridge flow rate |
| | | Isolate DNA | 1 ml/minute) |
| | | PCR reaction | C3a Assay |
| | | Total Time | Total Time 3.0 hours |
| | | 24 hours | Assumed 1% efficiency |
| | | Positive Reaction | Positive Reaction |
| 1,000 | 1,000 | Enrichment in | Positive Reaction |
| | | culture media | |
| | | Isolate DNA | |
| | | PCR reaction | |
| | | Total Time | |
| | | 24 hours | |
| | | Positive Reaction | |
| 10,000 | 10,000 | Filtration | Positive Reaction |
| | | Total Time | |
| | | 8 hours | |
| | | Positive Reaction | |

Above Time To Positive Detection (low contaminant numbers)

| | |
|---|---|
| 24 hours | 2.0 hours at 10% assay |
| at PCR assay efficiency | efficiency |
| | 3.0 hours at 1% assay |
| | efficiency |

ADVANTAGES OF MACMSA

Rapidity of contaminant detection
No enrichment in culture medium necessary
Detection at lower contamination levels by testing increased
volumes
Independence of enrichment techniques in media culture
*contamination 1 bacterium/1 ml

TABLE XII
CURRENT SENSITIVITY LEVELS FOR WATER
TESTING RANGE IN THE PARTS PER BILLION (PPB)
Example: Assume the target chemical's molecular
weight is 300 grams/molecule Gram Molecular
Weight = 300 grams/mole = 6.02 × 10²³
molecules

| | | Minimal Number Of Chemical |
|---|---|---|
| Parts Per | Definition | Molecules For Detection |
| Million (PPM) | 1 × 10⁻³ grams/liter or | (3.3 × 10⁻⁶ moles/liter) |
| | 1 milligram/liter | (6 × 10²³ molecules/mole) ≅ |
| | | 2 × 10¹⁸ molecules |
| Billion (PPB) | 1 × 10⁻⁶ grams/liter or | (3.3 × 10⁻⁹ moles/liter) |
| | 1 milligram/liter | (6 × 10²³ molecules/mole) ≅ |
| | | 2 × 10¹⁵ molecules |
| Trillion (PPT) | 1 × 10⁻⁹ grams/liter or | (3.3 × 10⁻¹² moles/liter) |
| | 1 milligram/liter | (6 × 10²³ molecules/mole) |
| | | ≅ 2 × 10¹² molecules or |
| | | 2,000,000,000,000 molecules |

MACMSA ANALYSIS ASSAY (using C4a)

| | Minimal Number Of Any |
|---|---|
| Assay | Immunogenic |
| Efficiency | Chemical Molecule Detected |
| 100% | >1 |
| 10% | >10 |
| 1% | >100 |

Compare to 2,000,000,000,000 molecules above

We claim:

1. A method of detecting a target analyte comprising the steps of:
providing a sample suspected of having a target analyte,
protecting a specific target analyte,
eliminating non-specific analytes, and
detecting the presence of target with a signal.

2. The method of claim 1 wherein the analyte is selected from nucleic acids.

3. The method of claim 1 wherein the analyte is selected from DNA, RNA, messenger RNA, ribosomal RNA, transfer RNA, viral RNA, and oncogenes.

4. A method of detecting a specific RNA target analyte comprising the steps of,
providing a sample suspected of having a target analyte,
forming a heterotriplex structure with the specific RNA target analyte and a DNA hairpin,
degrading non-specific RNA analytes, and
detecting the presence of target with a signal.

5. The method of claim 1 or 4 wherein the signal is a chemiluminescent signal.

6. A method of detecting a (pathologic) cell subset within a large (non-pathologic) cell population comprising the steps of,
providing a cell sample suspected of having a cell subset which possess specific immunogenic surface markers within said large non-pathologic, non-target cell population,
complexing a specific monoclonal antibody complementary to a specific target cell surface protein,
fixing the complexed monoclonal antibody and target cell surface protein complex and activating an immune complement cascade, and
detecting a product of the complement cascade as signal.

7. A method of detecting a soluble immunogenic target analyte comprising the steps of,
providing a sample suspected of having a soluble immunogenic target analyte, wherein said soluble immunogenic target analyte is selected from the group consisting of immunogenic proteins, peptides and chemicals, and combinations thereof,
complexing the soluble immunogenic target with a red blood cell stroma that has been sensitized by a monoclonal antibody specific to the soluble immunogenic target,
fixing the complexed monoclonal antibody, soluble immunogenic target and red blood cell stroma and activating an immune complement cascade, and
detecting a product of the complement cascade as a signal.

8. The method of claim 6 or 7 wherein the signal is a C3a peptide.

9. The method of claim 6 or 7 wherein the signal is a C4a peptide.

10. The method of claim 7 wherein the soluble immunogenic target is selected from a soluble peptide, protein, and immunogenic chemicals.

11. The method of claim 10 wherein the soluble immunogenic target is released from a cell cytoplasm or is released into a cellular environment.

12. The method of claims 1, 4, or 7 wherein the target analyte is sorted and separated from non-specific analyte.

13. The method of claim 6 wherein the cell subset is sorted and separated form non-specific cells.

14. A method of detecting an immunogenic target analyte comprising the steps of:
providing a sample suspected of having an immunogenic target analyte selected from the group consisting of cell surface particulate polysaccharide, lipopolysaccharide molecules, endotoxin, trypsin-like enzymes, and Ag/Ab complexes of IgA, and IgG4, that do not activate C1,
complexing the target analyte with specific complement components of the Alternate Pathway,
activating the Alternate Pathway at the C3 level, and
detecting a product of the Alternate Pathway as signal.

15. The method of claim 14 wherein the signal is generated at the C3 level and at levels subsequent to the C3 level of the Alternate Pathway.

16. The method of claim 15 wherein the signal is selected from the group consisting of C3a and C5a.
